SEQUENCE LISTING (15 sequences)

SEQ ID NO: 1 - protein, 448 aa, synthetic construct
  REGION 1..448: note = H1L1 Heavy Chain
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
  REGION 1..214: note = H1L1 Light Chain
DIQMTQSPATLSVSPGERATLSCKASQNVGGNVDWYQWKPGQAPRLLIYGASNRYTGIPARFRGSGSGTEFTLTISSLQSEDFAVYYCLQYKNNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 119 aa, synthetic construct
  REGION 1..119: note = H1L1 VH
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSS

SEQ ID NO: 4 - protein, 107 aa, synthetic construct
  REGION 1..107: note = H1L1 VL
DIQMTQSPATLSVSPGERATLSCKASQNVGGNVDWYQWKPGQAPRLLIYGASNRYTGIPARFRGSGSGTEFTLTISSLQSEDFAVYYCLQYKNNPWTFGQGTKVEIK

SEQ ID NO: 5 - protein, 8 aa, synthetic construct
  REGION 1..8: note = CDR H1
GFSFSSYG

SEQ ID NO: 6 - protein, 8 aa, synthetic construct
  REGION 1..8: note = CDR H2
ISPSGSYF

SEQ ID NO: 7 - protein, 9 aa, synthetic construct
  REGION 1..9: note = CDR H3
PGFNPPFDH

SEQ ID NO: 8 - protein, 6 aa, synthetic construct
  REGION 1..6: note = CDR L1
QNVGGN

SEQ ID NO: 9 - skipped

SEQ ID NO: 10 - protein, 9 aa, synthetic construct
  REGION 1..9: note = CDR L3
LQYKNNPWT

SEQ ID NO: 11 - protein, 448 aa, synthetic construct
  REGION 1..448: note = H3L3 Heavy Chain
QVQLVESGGGVVQPGRSLRLSCLGSGFSFSSYGMNWVRQAPGKGLEWVASISPSGSYFYYADSVKGRFTISRDNSKNTLYLQMTSLRAEDTAIYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 214 aa, synthetic construct
  REGION 1..214: note = H3L3 Light Chain
DIQMTQSPSSLSASVGDRVTITCKASQNVGGNVDWYQWKPGKAPKLLIYGASNRYTGVPDRFRGSGSGTDFTLTISSLQPEDVATYYCLQYKNNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 119 aa, synthetic construct
  REGION 1..119: note = H3L3 VH
QVQLVESGGGVVQPGRSLRLSCLGSGFSFSSYGMNWVRQAPGKGLEWVASISPSGSYFYYADSVKGRFTISRDNSKNTLYLQMTSLRAEDTAIYYCARLPGFNPPFDHWGQGTLVTVSS

SEQ ID NO: 14 - protein, 107 aa, synthetic construct
  REGION 1..107: note = H3L3 VL
DIQMTQSPSSLSASVGDRVTITCKASQNVGGNVDWYQWKPGKAPKLLIYGASNRYTGVPDRFRGSGSGTDFTLTISSLQPEDVATYYCLQYKNNPWTFGGGTKVEIK

SEQ ID NO: 15 - protein, 448 aa, synthetic construct
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0055179 A1
(43) Pub. Date: Feb. 26, 2026

(54) USE OF ANTI-CLAUDIN-1 ANTIBODIES TO TREAT CHOLANGIOPATHIES

(71) Applicants: Alentis Therapeutics AG, Basel (CH); Université de Strasbourg, Strasbourg (FR); Institut National de la Santé et de la Recherche Médicale, Paris (FR)

(72) Inventors: Thomas BAUMERT, Strasbourg (FR); Markus MEYER, Allschwil (CH); Roberto IACONE, Allschwil (CH); Alberto TOSO, Allschwil (CH); Tamás SCHWEIGHOFFER, Allschwil (CH); Geoffrey TEIXEIRA, Allschwil (CH); Fabio DEL ZOMPO, Strasbourg (FR)

(21) Appl. No.: 18/870,132

(22) PCT Filed: Jun. 1, 2023
(86) PCT No.: PCT/IB2023/055666

§ 371 Date: Nov. 27, 2024

Related U.S. Application Data
(60) Provisional application No. 63/365,679, filed on Jun. 1, 2022.

(51) Int. Cl. C07K 16/28; A61K 39/00; A61P 1/16

(52) U.S. Cl. CPC C07K 16/28; A61P 1/16; A61K 2039/505; C07K 2317/24

(57) ABSTRACT

The present disclosure relates to a method of a method of treating a cholangiopathy (e.g., Primary Sclerosing Cholangitis or Primary Biliary Cholangitis) in a human subject in need thereof, comprising administering a therapeutically effective amount of an anti-Claudin-1 antibody to the human subject.

### CROSS REFERENCE TO RELATED APPLICATIONS

This application is a U.S. National Phase Application, filed under 35 U.S.C. § 371(c), of International Application No PCT/IB2023/055666, filed Jun. 1, 2023, which claims the priority benefit of U.S. Provisional App. No. 63/365,679, filed Jun. 1, 2022, the contents of each of which are hereby incorporated by reference in its entirety.

### REFERENCE TO SEQUENCE LISTING SUBMITTED ELECTRONICALLY VIA EFS-WEB

The content of the electronically submitted sequence listing (Name: ALNT-008_N01US_SequenceListing_ST26; Size: 20,418 bytes; and Date of Creation: Nov. 5, 2025), filed with the application, is incorporated herein by reference in its entirety.

### FIELD OF THE INVENTION

According to various aspects of this disclosure, the present disclosure relates to methods of treating cholangiopathies.

### BACKGROUND OF THE INVENTION

Primary Sclerosing Cholangitis (PSC) is a chronic cholestatic disease characterized by peribiliary fibrosis leading to biliary strictures, end-stage liver disease and cholangiocarcinoma. Clinical care of PSC patients lacks effective therapeutic strategies other than liver transplantation. Claudin-1 (CLDN1) is a transmembrane protein involved in epithelial tight junctions and is also expressed non-junctionally, mediating cell plasticity and signaling. A need exists in the art for therapeutics targeting CLDN1 for the treatment of PSC.

### BRIEF SUMMARY OF THE INVENTION

The present disclosure provides a method of treating a cholangiopathy in a human subject in need thereof, comprising administering a therapeutically effective amount of an anti-Claudin-1 antibody to the human subject.

In some aspects, provided herein is a method of restoring the integrity of the bile duct epithelium in a human subject in need thereof, comprising administering a therapeutically effective amount of an anti-Claudin-1 antibody to the human subject. In some aspects, the administration results in modulation of hepatic progenitor cells and/or transdifferentiation of hepatocytes into ductal cells.

In some aspects, provided herein is a method to reduce cholestasis, improve liver inflammation and function.

In some aspects, provided herein is a method to improve survival from cholangiopathies.

In some aspects, provided herein is a method to prevent cholangiocarcinoma.

In some aspects, provided herein is a method of reducing PSC-associated ulcerative colitis, comprising administering an anti-Claudin-1 antibody to the human subject.

In some aspects, provided herein is a method of reducing biliary fibrosis, comprising administering an anti-Claudin-1 antibody to the human subject.

In some aspects, Claudin-1 (CLDN1) is overexpressed in the human subject compared to expression levels in a normal subject.

In some aspects, the anti-Claudin-1 antibody comprises the six complementary determining regions (CDRs) of an anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody is humanized.

In some aspects, the anti-Claudin-1 antibody comprises a VH comprising the amino acid sequence set forth in SEQ ID NO: 3 or SEQ ID NO: 13.

In some aspects, the anti-Claudin-1 antibody comprises a VL comprising the amino acid sequence set forth in SEQ ID NO: 4 or SEQ ID NO: 14.

In some aspects, the anti-Claudin-1 antibody comprises a VH comprising the amino acid sequence set forth in SEQ ID NO: 3; and a VL comprising the amino acid sequence set forth in SEQ ID NO: 4.

In some aspects, the anti-Claudin-1 antibody comprises a VH comprising the amino acid sequence set forth in SEQ ID NO: 13; and a VL comprising the amino acid sequence set forth in SEQ ID NO: 14.

In some aspects, the anti-Claudin-1 antibody comprises a heavy chain variable domain complementary determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7, and or a light chain variable domain complementary determining region (CDR) L1 comprising the amino acid sequence set forth in SEQ ID NO: 8, a CDR L2 comprising the amino acid sequence GAS, and a CDR L3 comprising the amino acid sequence set forth in SEQ ID NO: 10.

In some aspects, the anti-Claudin-1 antibody is administered intratumorally, intravenously, intraperitoneally, intramuscularly, intrathecally or subcutaneously.

In some aspects, provided herein is an anti-Claudin-1 antibody or a pharmaceutical composition thereof for use in a method of treating PSC in a human subject, the method comprising administering an effective amount of the anti-Claudin-1 antibody or a pharmaceutical composition thereof to the human subject.

In some aspects, the anti-Claudin-1 antibody for use in a method of treating PSC in a human subject comprises the six complementary determining regions (CDRs) of an anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody for use in a method of treating PSC in a human subject is humanized.

In some aspects, the anti-Claudin-1 antibody for use in a method of treating PSC in a human subject comprises a VH comprising the amino acid sequence set forth in SEQ ID NO: 3 or SEQ ID NO: 13.

In some aspects, the anti-Claudin-1 antibody for use in a method of treating PSC in a human subject comprises a VL comprising the amino acid sequence set forth in SEQ ID NO: 4 or SEQ ID NO: 14.

In some aspects, the anti-Claudin-1 antibody for use in a method of treating PSC in a human subject comprises a VH comprising the amino acid sequence set forth in SEQ ID NO: 3; and a VL comprising the amino acid sequence set forth in SEQ ID NO: 4.

In some aspects, the anti-Claudin-1 antibody for use in a method of treating PSC in a human subject comprises a VH comprising the amino acid sequence set forth in SEQ ID NO: 13; and a VL comprising the amino acid sequence set forth in SEQ ID NO: 14.

In some aspects, the anti-Claudin-1 antibody for use in a method of treating PSC in a human subject comprises a heavy chain variable domain complementary determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7, and/or a light chain variable domain complementary determining region (CDR) L1 comprising the amino acid sequence set forth in SEQ ID NO: 8, a CDR L2 comprising the amino acid sequence GAS, and a CDR L3 comprising the amino acid sequence set forth in SEQ ID NO: 10.

In some aspects, the anti-Claudin-1 antibody for use in a method of treating PSC in a human subject is administered intratumorally, intravenously, intraperitoneally, intramuscularly, intrathecally or subcutaneously.

In some aspects, provided herein is a kit for treating a subject suffering from a cholangiopathy, comprising a therapeutically effective amount of an anti-Claudin-1 antibody and an insert comprising instructions for use of the kit.

In some aspects, provided herein is a pharmaceutical composition for the treatment of a cholangiopathy, comprising a therapeutically effective amount of an anti-Claudin-1 antibody.

In some aspects, the cholangiopathy being treated by the method, use, kit, or pharmaceutical compositions disclosed herein is Primary Sclerosing Cholangitis (PSC).

In some aspects, the cholangiopathy being treated by the method, use, kit, or pharmaceutical compositions disclosed herein is Primary Biliary Cirrhosis (PBC)

In some aspects, the cholangiopathy being treated by the method, use, kit, or pharmaceutical compositions disclosed herein is a biliary fibrosis. In some aspects, the biliary fibrosis is caused by cystic fibrosis. In some aspects, the biliary fibrosis is caused by an IgG4-related disease.

In some aspects, the cholangiopathy is biliary atresia.

In some aspects, the cholangiopathy is Alagille syndrome.

### DESCRIPTION OF FIGURES

FIG. 1 (FIG. 1) shows an exemplary workflow for a study using 3,5-Diethoxycarbonyl-1,4-Dihydrocollidine (DDC) mouse model, a well-recognized animal model for PSC in which DDC was supplemented in the diet of C57BL/6 mice at 0.1%. Mice were treated with 25 mg/kg anti-Claudin-1 (CLDN1) monoclonal antibody or control weekly.

FIG. 2 (FIG. 2) shows an exemplary model of the mechanism of action of the therapeutic effect of anti-CLDN1 antibodies for PSC and other cholangiopathies.

FIGS. 3A-3D (FIGS. 3A-3D) show transcriptional profiling using bulk RNA-seq datasets deposited in the Gene Expression Omnibus, such as GSE20427 (FIG. 3A), GSE77503 (FIG. 3B), GSE28892 (FIG. 3C), and GSE29121 (FIG. 3D).

FIG. 4 (FIG. 4) shows biliary fibrosis in a DDC mouse model after treatment with control or anti-CLDN1 monoclonal antibody.

FIGS. 5A-5C (FIGS. 5A-5C) show fibrosis levels in a DDC mouse model after treatment with control or anti-CLDN1 monoclonal antibody. Fibrosis was measured by Sirius Red staining to determine the collagen proportionate area (CPA) (FIG. 5A), Ishak fibrosis score (FIG. 5B), and alkaline phosphatase levels (FIG. 5C).

FIGS. 6A-6C (FIGS. 6A-6C) show single-cell resolved transcriptomics studies analyzing Claudin-1 expression in healthy livers (FIG. 6A). FIG. 6B shows a violin plot displaying CLDN1 expression on the single cell level in cirrhotic tissue-derived liver cells compared to healthy liver. FIG. 6C shows that Claudin-1 co-clustered with known markers of biliary epithelial cells, such as BICC1, CYR61, DCDC2, EPCAM, ITGB8, KRT7, MAGI1, SLC5A1, and SOX9.

FIGS. 7A-7B (FIGS. 7A-7B) show immunohistochemistry of patient tissues showing co-expression of CLDN1 and CK19 in the surrounding ductular reaction (FIG. 7A) and co-expression of CLDN1 and EPCAM at the membrane of ductular reactive cells and hepatocytes (FIG. 7B).

FIG. 8 (FIG. 8) shows an exemplary workflow for a study using FRG-NOD Fah −/− mice engrafted with human hepatocytes or biliary cells to analyze the effect of anti-CLDN1 monoclonal antibody treatment in humanized mice on a DDC diet.

FIG. 9 (FIG. 9) shows an exemplary workflow for a study using bile duct ligation in a mouse model for cholangiopathies to analyze the effect of anti-CLDN1 monoclonal antibody treatment in models of cholestasis and biliary fibrosis.

FIG. 10 (FIG. 10) shows an exemplary workflow for a study using a Mdr2 −/− (Abcb4 −/−) mouse model that includes many features of PSC, including cholangiocarcinoma (CCA).

FIG. 11 (FIG. 11) shows an exemplary workflow for a study using HepaRG cell line to analyze the effect of anti-CLDN1 monoclonal antibody treatment on the fate of liver progenitor-like cells, hepatocytes and cholangiocytes.

FIGS. 12A-12C (FIGS. 12A-12C) show a more detailed exemplary workflow for the study in FIG. 11 using HepaRG cell line. FIG. 12A shows a differentiation workflow using Fluorescence activated cell sorting (FACS). FIG. 12B shows a de-differentiation workflow using FACS, immunofluorescence (IF), and RNA sequencing (RNA-seq). FIG. 12C shows a workflow for cholangiocyte analysis that utilizes RNA-seq.

FIG. 13A (FIG. 13A) shows the workflow of the experiment to investigate the dose-dependency of CLDN1 mAb treatment on biliary fibrosis in the DDC model (Fickert, P., et al., Am J Pathol. 171(2):525-36 (2007)). In the DDC model, a widely recognized model for PSC (Fickert, P., et al., J Hepatol. 60(6):1290-303. (2014)), 3,5-Diethoxycarbonyl-1,4-Dihydrocollidine was supplemented to animal chow at 0.1% for 5 days a week. 80 animals were randomized 1:1:1:1 in four groups, receiving either ALE.F02 CLDN1 mAb at 25 mg/kg, 10 mg/kg, 5 mg/kg (Roehlen, N., et al., Sci Transl Med. 14(676):eabj4221. (2022)), or vehicle control weekly. FIG. 13B (FIG. 13B) shows fibrosis levels in a DDC mouse model after treatment with control or increasing doses of anti-CLDN1 ALE.F02 monoclonal antibody. Fibrosis was measured by Sirius Red staining to determine the collagen proportionate area (CPA) as previously described (Roehlen et al. 2022). FIG. 13C (FIG. 13C) shows representative images of fibrosis in Sirius Red-stained livers from each group. Scale bars: 500 μm.

FIGS. 14A-14E (FIGS. 14A-14E) show transcriptomic analyses of CLDN1 expression in different cholangiopathies, based on analysis of microarray and bulk RNAseq datasets deposited in public repositories. These include E-GEOD-61260 (FIG. 14A and FIG. 14D for primary biliary cholangitis and primary sclerosing cholangitis, respectively), GSE46960 (FIG. 14B for biliary atresia), GSE206364 (FIG. 14C for Alagille syndrome), GSE118373 (FIG. 14E for ductular reactions in primary sclerosing cholangitis).

FIG. 15A (FIG. 15A) shows the workflow of the experiment performed in transgenic mice engineered to express a human/murine chimeric CLDN1 using the bile duct ligation model (BDL). Animals underwent surgical bile duct ligation, a widely used mouse model for obstructive cholestasis and secondary biliary fibrosis. Bile duct-ligated mice were assigned 1:1 to receive either 25 mg/kg CLDN1 H3L3 mAb (described in Colpitts, C C., et al., Gut67(4):736-745 (2018)) or vehicle control right after surgical ligation of the common bile duct and on day 4 after surgery. Surviving mice were euthanized on day 7 after surgery. Liver and plasma samples were harvested for subsequent analyses. FIG. 15B (FIG. 15B) shows the survival analysis of the two treatment groups, showing a survival advantage in CLDN1 mAb versus vehicle control-treated mice. FIGS. 15C-15E (FIGS. 15C-15E) show a marked and significant improvement of plasma levels of cholestasis and liver function tests in the CLDN1 mAb versus the control treated group. These include a reduction of plasma levels of alanine aminotransferase (ALT) (FIG. 15C), aspartate aminotransferase (AST) (FIG. 15C) as markers for liver inflammation as well as total bilirubin (FIG. 15D) and alkaline phosphatase as markers of cholestasis (FIG. 15E). Increased levels of albumin in CLDN1 mAb-versus control treated mice (FIG. 15D) demonstrate improvement of liver function by CLDN1 mAb antibody treatment. FIG. 15F (FIG. 15F) shows liver fibrosis levels after treatment with CLDN1 mAb or control, reduced by 36.8% in CLDN1 mAb-treated vs control mice (p<0.0001, Student's t-test). Fibrosis was measured by Sirius Red staining to determine the collagen proportionate area (CPA). FIG. 15G (FIG. 15G) shows liver fibrosis in representative sample of livers from control- and CLDN1 mAb-treated animals using Sirius Red-staining. Scale bars: 500 μm.

FIG. 16A (FIG. 16A) shows the experimental approach: To investigate the effect of CLDN1 mAb H3L3 treatment on the maturation of progenitor cells into mature hepatocyte-like cells, the HepaRG liver progenitor model was applied. FIG. 16B (FIG. 16B) shows the relative abundance of Na+-taurocholate cotransporting polypeptide protein positive (NTCP+) cells treated with H3L3 CLDN1 mAb (Colpitts et al. (2018)) or an isotype control during the differentiation protocol. NTCP protein is a marker of differentiated mature hepatocytes. Results from n=3 independent experiments are shown. FIG. 16C (FIG. 16C) shows mRNA expression of markers for mature hepatocytes in cells treated with CLDN1 mAb H3L3 versus isotype control Ab. Results from n=4 independent experiments are shown. Abbreviations: ALB—albumin; HNF4A—hepatocyte nuclear factor 4A; CYP3A4—cytochrome P450 3A4, TTR—trans-thyretin; UGT1A1-UDP Glucuronosyltransferase Family 1 Member A1; BAAT—bile acid-CoA:amino acid N-acyltransferase; TF—transferrin; SPP1—secreted phosphoprotein 1.

FIG. 17A (FIG. 17A) shows a graphical illustration of methodological approach: the effect of CLDN1 mAb treatment on gene expression was investigated in a cholangiocyte cell-based model treated with CLDN1 H3L3 or isotype control Ab. The cholangiocyte model consisted of HepaRG-differentiated cholangiocyte-like cells incubated with tumor necrosis factor α (TNF-α). Gene expression of treated cells was assessed by RNA-Seq and compared with gene expression and signaling pathways in the liver of PSC versus control patients described in (Horvath S., et al., Proc Natl Acad Sci USA 111(43):15538-15543 (2014))(E-GEOD-61260). FIG. 17B (FIG. 17B) shows modulation of inflammatory, fibrogenic, and oncogenic signaling pathways perturbed in PSC livers and restored after CLDN1 mAb treatment in cholangiocyte-like cells. Heat maps illustrate NES of altered gene sets (all FDR<0.05).

FIG. 18A (FIG. 18A) shows CLDN1 expression in untreated and TNF-α treated primary human cholangiocytes. CLDN1 protein targeting by CLDN1 mAb H3L3 on human cholangiocytes was demonstrated by flow cytometry. AMFI (mean fluorescence intensity) is shown as fold change compared with untreated cells.

FIGS. 18B and 18C (FIGS. 18B-18C) show the effect of treatment of primary human cholangiocytes on TNF-α-NFkB signaling. FIG. 18B shows representative immunoblots of pIKBα, IKBα, and GAPDH and respective protein quantification (n=10, p=0.003, Mann-Whitney U test) in cholangiocytes treated with CLDN1 H3L3 (Colpitts et al. (2018)) or an isotype control mAb following TNFα stimulation. FIG. 18C shows representative immunoblots of pP65, P65, and GAPDH and respective protein quantification (n=10; p=0.0002, Mann-Whitney U test) in cholangiocytes treated with CLDN1 H3L3 or isotype control mAb and following TNFα stimulation. Bars express normalized phosphorylated/total protein ratios. Error bars express standard error of the mean.

### DETAILED DESCRIPTION OF THE INVENTION

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “treating” or “treatment” as used herein refers to the administration of a composition to a subject for therapeutic purposes.

The term “human Claudin-1 (or CLDN1)” refers to a protein having the sequence shown in NCBI Accession Number NP_066924.1, or any naturally occurring variants commonly found in HCV permissive human populations.

The term “antibody”, as used herein, refers to any immunoglobulin that contains an antigen binding site that immunospecifically binds an antigen. As such, the term antibody encompasses not only whole antibody molecules, but also antibody fragments as well as variants (including derivatives) of antibodies and of antibody fragments as long as the derivatives and fragments maintain specific binding ability. The term encompasses monoclonal antibodies and polyclonal antibodies. The term also covers any protein having a binding domain, which is homologous or largely homologous to an immunoglobulin-binding domain. These proteins may be derived from natural sources, or partly or wholly synthetically produced. The term “specific binding”, when used in reference to an antibody, refers to an antibody binding to a predetermined antigen. Typically, the antibody binds with an affinity of at least 1×10⁷ M¹, and binds to the predetermined antigen with an affinity that is at least two-fold greater than the affinity for binding to a non-specific antigen (e.g., BSA, casein).

As used herein, the term “humanized antibody” refers to a chimeric antibody comprising amino acid residues from non-human hypervariable regions and amino acid residues from human framework regions (FRs). In particular, a humanized antibody comprises all or substantially all of at least one, typically two, variable domains, in which all or substantially all of the complementarity determining regions (CDRs) are those of a human antibody. A humanized antibody optionally may comprise at least a portion of an antibody constant region derived from a human antibody. A “humanized form” of an antibody, e.g., a non-human antibody, refers to an antibody that has undergone humanization.

It is understood that wherever aspects are described herein with the language “comprising,” otherwise analogous aspects described in terms of “consisting of” and/or “consisting essentially of” are also provided.

As used herein, the term “administering” refers to the physical introduction of a composition comprising a therapeutic agent (e.g., an anti-Claudin-1 antibody) to a subject, using any of the various methods and delivery systems known to those skilled in the art. Routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit (i.e., slow to some extent and may stop tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of anti-Claudin-1 antibody clinically proven to affect a significant decrease in cancer or slowing of progression of cancer, such as an advanced solid tumor.

A “patient” as used herein includes any patient who is afflicted with a cancer (e.g., a fibrotic cancer). The terms “subject” and “patient” are used interchangeably herein.

### II. Anti-Claudin-1 Antibodies

The present invention concerns the use of anti-Claudin-1 antibodies for the treatment of a cholangiopathy. In some aspects, the cholangiopathy is PSC. In some aspects, the cholangiopathy is PBC. In some aspects, the cholangiopathy is a genetic cholangiopathy. In some aspects, the cholangiopathy is an idiopathic cholangiopathy. In some aspects, the cholangiopathy is a malignant cholangiopathy. In some aspects, the cholangiopathy is a secondary sclerosing cholangitis.

In some aspects, the genetic cholangiopathies are selected from the group consisting of Alagille syndrome, Caroli syndrome, Cystic fibrosis, Progressive Familial Intrahepatic Cholestasis, and Polycystic liver disease.

In some aspects, the idiopathic cholangiopathies are selected from the group consisting of Autoimmune cholangitis, Biliary atresia, Idiopathic childhood or adulthood ductopenia, IgG4-associated cholangitis, Primary biliary cirrhosis, and Primary sclerosing cholangitis.

In some aspects, the malignant cholangiopathy is cholangiocarcinoma.

In some aspects, the secondary sclerosing cholangitis is selected from the group consisting of ABCB4 deficiency, abdominal trauma (surgical or blunt), AIDS cholangiopathy, amyloidosis, chemical/drugs (i.e., 5-fluorouracil), choledocholithiasis, eosinophilic or mast cell cholangitis, graft-vs-host disease involving the liver, iatrogenic biliary strictures, portal hypertensive biliopathy, recurrent pyogenic cholangitis, sarcoidosis, sickle cell disease, and vascular/ischemic (i.e., hepatic artery stenosis after liver transplant).

CLDN1 is a transmembrane protein with two major roles: (1) together with other proteins it contributes to the barrier function by tight junctions; (2) it is expressed outside the tight junctions in the basolateral membrane of epithelial cells, where CLDN1 has been shown to mediate procarcinogenic signaling, epithelial-mesenchymal transition (EMT) and cell fate. Moreover, it has been shown that CLDN1 is also expressed by nonepithelial cells such as myofibroblasts of liver, lung, and kidney.

Antibodies directed against human Claudin-1 have been previously described to treat hepatitis C virus infection, hepatocellular carcinoma, and certain fibrotic diseases, such as lung fibrosis (see WO 2010/034812, WO 2016/146809, and WO 2021/094469). Anti-Claudin-1 antibodies that can be used in the practice of the present invention include any antibody raised against Claudin-1. Examples are disclosed in WO 2010/034812 and WO 2017/162678.

Other examples of suitable anti-Claudin-1 antibodies include those disclosed in European Patent No. EP 1 167 389, in U.S. Pat. No. 6,627,439, in international patent application published under No. WO 2014/132307, in international patent applications published under No. WO 2015/014659 and No. WO 2015/014357, and in Yamashita et al., J. Pharmacol. Exp. Ther., 2015, 353(1): 112-118.

Anti-Claudin-1 antibodies suitable for use in the present invention may be polyclonal antibodies or monoclonal antibodies.

Anti-Claudin-1 antibodies suitable for use according to the present invention may also be “humanized”: sequence differences between rodent antibodies and human sequences can be minimized by replacing residues which differ from those in the human sequences by site-directed mutagenesis of individual residues or by grafting of entire regions or by chemical synthesis. Humanized antibodies can also be produced using recombinant methods. In the humanized form of the antibody, some, most or all of the amino acids outside the CDR regions are replaced with amino acids from human immunoglobulin molecules, while some, most or all amino acids within one or more CDR regions are unchanged. Small additions, deletions, insertions, substitutions or modifications of amino acids are permissible as long as they do not significantly modify the biological activity of the resulting antibody. Suitable human “replacement” immunoglobulin molecules include IgG1, IgG2, IgG2a, IgG2b, IgG3, IgG4, IgA, IgM, IgD or IgE molecules, and fragments thereof.

In some aspects, a humanized anti-Claudin-1 antibody for use according to the present invention is one previously described in WO 2017/162678. Exemplary sequences for the antibody or antigen binding fragment provided herein are described in Table 1.

TABLE 1
Exemplary Sequences

| | | SEQ ID |
|---|---|---|
| Description | Sequence | NO: |
| H1L1- | EVQLVESGGGLVKPGGSLRLSCAASGFSFSSYGMNWVRQA | 1 |
| Heavy | PGKGLEWVSSISPSGSYFYYADSVKGRFTISRDNAKNSLYLQ | |
| Chain #1 | MNSLRAEDTAVYYCARLPGFNPPFDHWGQGTLVTVSSAST | |
| (ALE.F02) | KGPSVFPLAPSSKSTSGGTAALGCLVKDYFPEPVTVSWNSG | |
| | ALTSGVHTFPAVLQSSGLYSLSSVVTVPSSSLGTQTYICNVN | |
| | HKPSNTKVDKRVEPKSCDKTHTCPPCPAPEFEGGPSVFLFPP | |
| | KPKDTLMISRTPEVTCVVVDVSHEDPEVKFNWYVDGVEVH | |
| | NAKTKPREEQYNSTYRVVSVLTVLHQDWLNGKEYKCKVS | |
| | NKALPASIEKTISKAKGQPREPQVYTLPPSREEMTKNQVSLT | |
| | CLVKGFYPSDIAVEWESNGQPENNYKTTPPVLDSDGSFFLY | |
| | SKLTVDKSRWQQGNVFSCSVMHEALHNHYTQKSLSLSPG | |
| H1L1- | DIQMTQSPATLSVSPGERATLSCKASQNVGGNVDWYQWKP | 2 |
| Light Chain | GQAPRLLIYGASNRYTGIPARFRGSGSGTEFTLTISSLQSEDF | |
| | AVYYCLQYKNNPWTFGQGTKVEIKRTVAAPSVFIFPPSDEQ | |
| | LKSGTASVVCLLNNFYPREAKVQWKVDNALQSGNSQESVT | |
| | EQDSKDSTYSLSSTLTLSKADYEKHKVYACEVTHQGLSSPV | |
| | TKSFNRGEC | |
| H1L1-VH | EVQLVESGGGLVKPGGSLRLSCAASGFSFSSYGMNWVRQA | 3 |
| | PGKGLEWVSSISPSGSYFYYADSVKGRFTISRDNAKNSLYLQ | |
| | MNSLRAEDTAVYYCARLPGFNPPFDHWGQGTLVTVSS | |
| H1L1-VL | DIQMTQSPATLSVSPGERATLSCKASQNVGGNVDWYQWKP | 4 |
| | GQAPRLLIYGASNRYTGIPARFRGSGSGTEFTLTISSLQSEDF | |
| | AVYYCLQYKNNPWTFGQGTKVEIK | |
| H1L1/H3L3- | GFSFSSYG | 5 |
| CDR H1 | | |
| H1L1/H3L3- | ISPSGSYF | 6 |
| CDR H2 | | |
| H1L1/H3L3- | PGFNPPFDH | 7 |
| CDR H3 | | |
| H1L1/H3L3- | QNVGGN | 8 |
| CDR L1 | | |
| H1L1/H3L3- | GAS | — |
| CDR L2 | | |
| H1L1/H3L3- | LQYKNNPWT | 10 |
| CDR L3 | | |
| H3L3- | QVQLVESGGGVVQPGRSLRLSCLGSGFSFSSYGMNWVRQA | 11 |
| Heavy | PGKGLEWVASISPSGSYFYYADSVKGRFTISRDNSKNTLYLQ | |
| Chain | MTSLRAEDTAIYYCARLPGFNPPFDHWGQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVNH | |
| | KPSNTKVDKRVEPKSCDKTHTCPPCPAPELLGGPSVFLFPPK | |
| | PKDTLMISRTPEVTCVVVDVSHEDPEVKFNWYVDGVEVHN | |
| | AKTKPREEQYNSTYRVVSVLTVLHQDWLNGKEYKCKVSN | |
| | KALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVFSCSVMHEALHNHYTQKSLSLSPG | |
| H3L3- | DIQMTQSPSSLSASVGDRVTITCKASQNVGGNVDWYQWKP | 12 |
| Light Chain | GKAPKLLIYGASNRYTGVPDRFRGSGSGTDFTLTISSLQPED | |
| | VATYYCLQYKNNPWTFGGGTKVEIKRTVAAPSVFIFPPSDE | |
| | QLKSGTASVVCLLNNFYPREAKVQWKVDNALQSGNSQESV | |
| | TEQDSKDSTYSLSSTLTLSKADYEKHKVYACEVTHQGLSSP | |
| | VTKSFNRGEC | |
| H3L3-VH | QVQLVESGGGVVQPGRSLRLSCLGSGFSFSSYGMNWVRQA | 13 |
| | PGKGLEWVASISPSGSYFYYADSVKGRFTISRDNSKNTLYLQ | |
| | MTSLRAEDTAIYYCARLPGFNPPFDHWGQGTLVTVSS | |
| H3L3-VL | DIQMTQSPSSLSASVGDRVTITCKASQNVGGNVDWYQWKP | 14 |
| | GKAPKLLIYGASNRYTGVPDRFRGSGSGTDFTLTISSLQPED | |
| | VATYYCLQYKNNPWTFGGGTKVEIK | |
| H1L1 | EVQLVESGGGLVKPGGSLRLSCAASGFSFSSYGMNWVRQA | 15 |
| Heavy | PGKGLEWVSSISPSGSYFYYADSVKGRFTISRDNAKNSLYLQ | |
| Chain #2 | MNSLRAEDTAVYYCARLPGFNPPFDHWGQGTLVTVSSAST | |
| (ALE.C04) | KGPSVFPLAPSSKSTSGGTAALGCLVKDYFPEPVTVSWNSG | |
| | ALTSGVHTFPAVLQSSGLYSLSSVVTVPSSSLGTQTYICNVN | |
| | HKPSNTKVDKRVEPKSCDKTHTCPPCPAPELLGGPSVFLFPP | |
| | KPKDTLMISRTPEVTCVVVDVSHEDPEVKFNWYVDGVEVH | |
| | NAKTKPREEQYNSTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLY | |
| | SKLTVDKSRWQQGNVFSCSVMHEALHNHYTQKSLSLSPG | |

In some aspects, the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

In some aspects, the anti-Claudin-1 antibody comprises a complementarity determining region (CDR) L1 comprising the amino acid sequence set forth in SEQ ID NO: 8, a CDR L2 comprising the amino acid sequence set forth as GAS, and a CDR L3 comprising the amino acid sequence set forth in SEQ ID NO: 10.

In some aspects, the complementarity determining regions (CDRs) disclosed herein are defined according to IMGT®. However, it is appreciated that other methods of defining the CDRs in the art can also be used.

In some aspects, the six complementarity determining regions (CDRs) of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the heavy chain variable region (“VH”) and the light chain variable region (“VL”) of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody comprises a VH comprising the amino acid sequence set forth in SEQ ID NO: 3 or SEQ ID NO: 13.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 3.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 13.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 3.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 13.

In some aspects, the anti-Claudin-1 antibody comprises a VL comprising the amino acid sequence set forth in SEQ ID NO: 4 or SEQ ID NO: 14.

In some aspects, the anti-Claudin-1 antibody comprises a VL comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 4.

In some aspects, the anti-Claudin-1 antibody comprises a VL comprising an amino acid sequence having at least 85%, at least 90%, at least 91%, at least 92%, at least 93%, at least 94%, at least 95%, at least 96%, at least 97%, at least 98%, at least 99% sequence identity to SEQ ID NO: 14.

In some aspects, the anti-Claudin-1 antibody comprises a VL comprising an amino acid sequence having about 85%, about 90%, about 91%, about 92%, about 93%, about 94%, about 95%, about 96%, about 97%, about 98%, about 99%, or about 100% sequence identity to SEQ ID NO: 4.

In some aspects, the anti-Claudin-1 antibody comprises a VL comprising an amino acid sequence having about 85%, about 90%, about 91%, about 92%, about 93%, about 94%, about 95%, about 96%, about 97%, about 98%, about 99%, or about 100% sequence identity to SEQ ID NO: 14.

In some aspects, the anti-Claudin-1 antibody comprises a VH comprising the amino acid sequence set forth in SEQ ID NO: 3; and a VL comprising the amino acid sequence set forth in SEQ ID NO: 4.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 3 and a light chain variable region (VL) comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 4.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 3 and a light chain variable region (VL) comprising an amino acid sequence having about 85%, about 90%, about 91%, about 92%, about 93%, about 94%, about 95%, about 96%, about 97%, about 98%, about 99%, or about 100% sequence identity to SEQ ID NO: 4.

In some aspects, the anti-Claudin-1 antibody comprises a VH comprising the amino acid sequence set forth in SEQ ID NO: 13; and a VL comprising the amino acid sequence set forth in SEQ ID NO: 14.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 13 and a light chain variable region (VL) comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 14.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 13 and a light chain variable region (VL) comprising an amino acid sequence having about 85%, about 90%, about 91%, about 92%, about 93%, about 94%, about 95%, about 96%, about 97%, about 98%, about 99%, or about 100% sequence identity to SEQ ID NO: 14.

In some aspects, the heavy chain and light chain of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody comprises a heavy chain comprising the amino acid sequence set forth in SEQ ID NO: 1.

In some aspects, the anti-Claudin-1 antibody comprises a heavy chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 1.

In some aspects, the anti-Claudin-1 antibody comprises a heavy chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 1.

In some aspects, the anti-Claudin-1 antibody comprises a light chain comprising the amino acid sequence set forth in SEQ ID NO: 2.

In some aspects, the anti-Claudin-1 antibody comprises a light chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 2.

In some aspects, the anti-Claudin-1 antibody comprises a light chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 2.

In some aspects, the anti-Claudin-1 antibody comprises a heavy chain comprising the amino acid sequence set forth in SEQ ID NO: 1; and a light chain comprising the amino acid sequence set forth in SEQ ID NO: 2.

In some aspects, the anti-Claudin-1 antibody comprises a heavy chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 1 and a light chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 2.

In some aspects, the anti-Claudin-1 antibody comprises a heavy chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 1 and a light chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 2.

In some aspects, the anti-Claudin-1 antibody comprises a heavy chain comprising the amino acid sequence set forth in SEQ ID NO: 11.

In some aspects, the anti-Claudin-1 antibody comprises a heavy chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 11.

In some aspects, the anti-Claudin-1 antibody comprises a heavy chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 11.

In some aspects, the anti-Claudin-1 antibody comprises a light chain comprising the amino acid sequence set forth in SEQ ID NO: 12.

In some aspects, the anti-Claudin-1 antibody comprises a light chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 12.

In some aspects, the anti-Claudin-1 antibody comprises a light chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 12.

In some aspects, the anti-Claudin-1 antibody comprises a heavy chain comprising the amino acid sequence set forth in SEQ ID NO: 11; and a light chain comprising the amino acid sequence set forth in SEQ ID NO: 12.

In some aspects, the anti-Claudin-1 antibody comprises a heavy chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 11 and a light chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 12.

In some aspects, the anti-Claudin-1 antibody comprises a heavy chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 11 and a light chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 12.

The humanized anti-Claudin-1 antibody may be a full monoclonal antibody having an isotope selected from the group consisting of IgG1, IgG2, IgG3 and IgG4. Alternatively, the humanized anti-Claudin-1 antibody may be a fragment of a monoclonal antibody selected from the group consisting of Fv, Fab, F(ab′)2, Fab′, dsFv, scFv, sc(Fv)₂ and diabodies.

Anti-Claudin-1 antibodies (or biologically active variants or fragments thereof) suitable for use according to the present invention may be functionally linked (e.g., by chemical coupling, genetic fusion, non-covalent association or otherwise) to one or more other molecular entities. Methods for the preparation of such modified antibodies (or conjugated antibodies) are known in the art (see, for example, “Affinity Techniques. Enzyme Purification: Part B”, Methods in Enzymol., 1974, Vol. 34, Jakoby and Wilneck (Eds.), Academic Press: New York, NY; and Wilchek and Bayer, Anal. Biochem., 1988, 171: 1-32). Preferably, molecular entities are attached at positions on the antibody molecule that do not interfere with the binding properties of the resulting conjugate, e.g., positions that do not participate in the specific binding of the antibody to its target.

In some aspects, the anti-Claudin-1 antibodies described herein target the extracellular loop 1 of exposed Claudin-1 outside of tight junctions in the basolateral membrane of epithelial cells (as described in Mailly L et al. Nature Biotech 2015).

The antibody molecule and molecular entity may be covalently, directly linked to each other. Or, alternatively, the antibody molecule and molecular entity may be covalently linked to each other through a linker group. This can be accomplished by using any of a wide variety of stable bifunctional agents well known in the art, including homofunctional and heterofunctional linkers.

In some aspects, an anti-Claudin-1 antibody (or a biologically active fragment thereof) for use according to the present invention is conjugated to a detectable agent. Any of a wide variety of detectable agents can be used, including, without limitation, various ligands, radionuclides (e.g., ³H, ¹²⁵I, ¹³¹I, and the like), fluorescent dyes (e.g., fluorescein isothiocyanate, rhodamine, phycoerytherin, phycocyanin, allophycocyanin, o-phthalaldehyde and fluorescamine), chemiluminescent agents (e.g., luciferin, luciferase and aequorin), microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

Other molecular entities that can be conjugated to an anti-Claudin-1 antibody of the present invention (or a biologically active fragment thereof) include, but are not limited to, linear or branched hydrophilic polymeric groups, fatty acid groups, or fatty ester groups.

Thus, in the practice of the present invention, anti-Claudin-1 antibodies can be used under the form of full length antibodies, biologically active variants or fragments thereof, chimeric antibodies, humanized antibodies, and antibody-derived molecules comprising at least one complementarity determining region (CDR) from either a heavy chain or light chain variable region of an anti-Claudin-1 antibody, including molecules such as Fab fragments, F(ab′)2 fragments, Fd fragments, Fabc fragments, Sc antibodies (single chain antibodies), diabodies, individual antibody light single chains, individual antibody heavy chains, chimeric fusions between antibody chains and other molecules, and antibody conjugates, such as antibodies conjugated to a therapeutic agent or a detectable agent. Preferably, anti-Claudin-1 antibody-related molecules according to the present invention retain the antibody's ability to bind its antigen, in particular the extracellular domain of Claudin-1.

### III. Cholangiopathies

Cholangiopathies are a heterogeneous group of diseases affecting the biliary tree, either in its intra- and extrahepatic part. Whereas rare when taken individually, on aggregate they represent up to 80% of liver transplantation indications in the pediatric age, as well as up to 20% of them in the adult age. The cholangiopathies include primary biliary cholangitis or primary biliary cirrhosis (PBC), and primary sclerosing cholangitis (PSC). Cholangiopathies may be genetic, idiopathic, malignant, or secondary sclerosing cholangitis. Genetic cholangiopathies include Alagille syndrome, Caroli syndrome, Cystic fibrosis, and polycystic liver disease. Idiopathic cholangiopathies include autoimmune cholangitis, biliary atresia, idiopathic childhood or adulthood ductopenia, IgG4-associated cholangitis, primary biliary cirrhosis, and primary sclerosing cholangitis. Malignant cholangiopathies include cholangiocarcinoma. Secondary sclerosing cholangitis include ABCB4 deficiency, Abdominal trauma (surgical or blunt), AIDS cholangiopathy, Amyloidosis, chemical/drugs (i.e., 5-fluorouracil), choledocholithiasis, eosinophilic or mast cell cholangitis, graft-vs-host disease involving the liver, iatrogenic biliary strictures, portal hypertensive biliopathy, recurrent pyogenic cholangitis, sarcoidosis, sickle cell disease, clonorchis sinensis infestation, post-viral sclerosing cholangitis (i.e. after COVID-19 infection), extrinsic obstructions of the biliary tree (e.g. benign and malignant lympadenopathies, extrahepatic biliary tract tumors, pancreatic tumors and ampullomas) and vascular/ischemic (i.e., hepatic artery stenosis after liver transplant).

PSC has an estimated prevalence of 16.2 cases every 100.000 people in Europe. PSC is a rare chronic cholestatic liver disease characterized by inflammatory destruction of the intrahepatic and/or extrahepatic bile ducts, leading to bile stasis, fibrosis, and ultimately to cirrhosis, and often requires liver transplantation (LT). Most cases occur in association with inflammatory bowel disease (IBD), which often precedes the development of PSC. PSC is usually diagnosed after detection of cholestasis during health evaluation or screening of patients with IBD (see Rabiee A, Silveira M G; Primary sclerosing cholangitis; Transl Gastroenterol Hepatol; vol. 6:29 (Apr. 5 2021)).

In patients who present with symptoms, abdominal pain is the most frequent symptom (20%) followed by pruritus (10%), jaundice (6%), and fatigue (6%) (3), but the presentation may differ widely among patients. Hepatomegaly and splenomegaly can be present in 44% and 39% of patients, respectively. Acute pruritus and/or cholangitis, presenting with jaundice, fever, and abdominal pain, may be a result of benign or malignant biliary tract obstruction. Indeed, worsening cholestatic signs and symptoms should raise concern about cholangiocarcinoma (CCA), the most feared and not uncommon complication of PSC. Presentation with variceal bleeding, ascites, or hepatic encephalopathy may occur once a patient has progression to end-stage liver disease. In patients with associated IBD, abdominal pain, diarrhea, and gastrointestinal bleeding may be the only presenting symptoms along with abnormal liver biochemistries. Elevations in serum alkaline phosphatase (ALP) and gamma-glutamyl transferase values in a cholestatic pattern are the biochemical hallmark of PSC, though up to 30% to 40% of patients have normal ALP at diagnosis or during the course of their disease. Increases of serum aspartate and alanine aminotransferase levels are usually less pronounced and typically less than 5 times the upper limits of normal (ULN). The serum total bilirubin level is normal in the majority of cases at diagnosis. (Silveria, Transl Gastroenterol Hepatol; vol. 6:29 (Apr. 5 2021)).

One histological hallmark of PSC is the finding of concentric periductal fibrosis, also known as “onion-skin fibrosis”, but it is only detected in less than 15% of liver biopsies of patients with PSC. (Silveria, Transl Gastroenterol Hepatol; vol. 6:29 (Apr. 5 2021)).

Following disease progression, fibrotic strictures of both intra- and extrahepatic bile ducts impair the function of the biliary tree, leading to obstructive jaundice with untreatable pruritus, an increased risk of life-threatening bacterial infections (i.e. cholangitis), and development of liver cirrhosis with end-stage liver disease. Up to 20% of PSC patients develop cholangiocarcinoma, thus PSC itself is considered as a pre-malignant disease.

A hallmark of cholangiopathies such as PSC is the ductular reaction. Upon chronic intrahepatic cholangiocyte damage, integrity of the bile duct epithelium is restored through expansion of hepatic progenitor cells and/or transdifferentiation of hepatocytes into ductal cells. Reactive ductules and activated myofibroblasts sustain each other in a two-way fashion. In a previous transcriptomic analysis of the ductular reaction of liver tissues of patients with HCV infection and PSC, CLDN1 was among the 20 top upregulated genes in PSC vs HCV-induced liver disease, suggesting a potential disease association of CLDN1 and biliary injury.

Primary biliary cholangitis (PBC), also known as primary biliary cirrhosis, is a chronic cholestatic liver disease. PBC is a multifactorial and enigmatic disease. Autoimmune attacks targeted at biliary epithelial cells through tolerance breakdown triggers disease onset.

The hallmark of PBC is anti-mitochondrial antibody, which are detected in 90-95% of patients with PBC, and the most disease-specific autoantibodies in human immunopathology. Anti-mitochondrial antibodies recognize a family of enzymes located in the inner membrane of the mitochondria, named the 2-oxo-acid dehydrogenase complex (2-OADC), which mainly includes the pyruvate dehydrogenase complex E2 subunit (PDC-E2), the branched-chain 2-OADC E2 subunit (BCOADC-E2), the 2-oxoglutaric acid dehydrogenase complex E2 subunit (OGDC-E2), and dihydrolipoamide dehydrogenase-binding protein (E3BP). Anti-mitochondrial antibodies and autoreactive CD4+ and CD8+ T cell epitopes are confined within a shared peptide sequence of the inner lipoyl domain of human PDC-E2.

Biliary epithelial cells and hepatocytes of patients with PBC express large amounts of human leukocyte antigen (HLA) classes I and II molecules. Biliary epithelial cells express adhesion molecules, cytokines, and chemokines, and recruit mononuclear cells in the biliary tract of the liver. One example is fractalkine (CX3CL1), a chemokine with both chemoattractant and cell-adhesive functions. T helper type 1-cytokine predominance and lipopolysaccharide in the microenvironment of injured bile ducts induce the upregulation of fractalkine expression in biliary epithelial cells, followed by the chemoattraction of mononuclear cells expressing its receptor (CX3CR1), including CD4+ and CD8+ T cells. Serum fractalkine levels in PBC are high in patients with marked cholangitis activity (CA) at early stages, and they decrease in response to treatment.

In some aspects, the cholangiopathy is biliary atresia.

In some aspects, the cholangiopathy is Alagille syndrome.

Other factors related to PBC progression include genetic predisposition, environmental triggers, and microbiota composition.

### IV. Methods of Use

Methods of the present invention may be accomplished using an anti-Claudin-1 antibody, or a biologically active fragment thereof described herein, or a pharmaceutical composition comprising such an antibody or fragment. These methods generally comprise administration of an effective amount of an anti-Claudin-1 antibody, or biologically active fragment thereof, or of a pharmaceutical composition thereof, to a subject in need thereof (i.e., a subject having a fibrotic tumor). Administration can be performed using any of the administration methods known to one skilled in the art.

The present disclosure provides a method of treating a cholangiopathy in a human subject in need thereof, comprising administering a therapeutically effective amount an anti-Claudin-1 antibody to the human subject.

In some aspects, the cholangiopathy is Primary Sclerosing Cholangitis (PSC).

In some aspects, the cholangiopathy is Primary Biliary Cirrhosis (PBC) (also called Primary Biliary Cholangitis).

In some aspects, the cholangiopathy is a genetic cholangiopathy. In some aspects, the cholangiopathy is an idiopathic cholangiopathy. In some aspects, the cholangiopathy is a malignant cholangiopathy. In some aspects, the cholangiopathy is a secondary sclerosing cholangitis.

In some aspects, the genetic cholangiopathies are selected from the group consisting of Alagille syndrome, Caroli syndrome, cystic fibrosis, and polycystic liver disease.

In some aspects, the idiopathic cholangiopathies are selected from the group consisting of autoimmune cholangitis, biliary atresia, idiopathic childhood or adulthood ductopenia, IgG4-associated cholangitis, primary biliary cirrhosis, and primary sclerosing cholangitis.

In some aspects, the malignant cholangiopathy is cholangiocarcinoma.

In some aspects, the secondary sclerosing cholangitis is selected from the group consisting of ABCB4 deficiency, Abdominal trauma (surgical or blunt), AIDS cholangiopathy, Amyloidosis, chemical/drugs (i.e., 5-fluorouracil), choledocholithiasis, eosinophilic or mast cell cholangitis, graft-vs-host disease involving the liver, iatrogenic biliary strictures, portal hypertensive biliopathy, recurrent pyogenic cholangitis, sarcoidosis, sickle cell disease, and vascular/ischemic (i.e., hepatic artery stenosis after liver transplant).

In some aspects, provided herein is a method of restoring the integrity of the bile duct epithelium in a human subject in need thereof, comprising administering a therapeutically effective amount of an anti-Claudin-1 antibody to the human subject. In some aspects, the administration results in expansion of hepatic progenitor cells and/or transdifferentiation of hepatocytes into ductal cells.

In some aspects, provided herein is a method of reducing PSC-associated ulcerative colitis, comprising administering an anti-Claudin-1 antibody to the human subject.

In some aspects, provided herein is a method of reducing Biliary Fibrosis, comprising administering an anti-Claudin-1 antibody to the human subject.

In some aspects, Claudin-1 (CLDN1) is overexpressed in the human subject compared to expression levels in a normal subject.

In some aspects, the anti-Claudin-1 antibody comprises the six complementary determining regions (CDRs) of an anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody is humanized.

In some aspects, the anti-Claudin-1 antibody comprises a VH comprising the amino acid sequence set forth in SEQ ID NO: 3 or SEQ ID NO: 13.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 3.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 13.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 3.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 13.

In some aspects, the anti-Claudin-1 antibody comprises a VL comprising the amino acid sequence set forth in SEQ ID NO: 4 or SEQ ID NO: 14.

In some aspects, the anti-Claudin-1 antibody comprises a VL comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 4.

In some aspects, the anti-Claudin-1 antibody comprises a VL comprising an amino acid sequence having at least 85%, at least 90%, at least 91%, at least 92%, at least 93%, at least 94%, at least 95%, at least 96%, at least 97%, at least 98%, at least 99% sequence identity to SEQ ID NO: 14.

In some aspects, the anti-Claudin-1 antibody comprises a VL comprising an amino acid sequence having about 85%, about 90%, about 91%, about 92%, about 93%, about 94%, about 95%, about 96%, about 97%, about 98%, about 99%, or about 100% sequence identity to SEQ ID NO: 4.

In some aspects, the anti-Claudin-1 antibody comprises a VL comprising an amino acid sequence having about 85%, about 90%, about 91%, about 92%, about 93%, about 94%, about 95%, about 96%, about 97%, about 98%, about 99%, or about 100% sequence identity to SEQ ID NO: 14.

In some aspects, the anti-Claudin-1 antibody comprises a VH comprising the amino acid sequence set forth in SEQ ID NO: 3; and a VL comprising the amino acid sequence set forth in SEQ ID NO: 4.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 3 and a light chain variable region (VL) comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 4.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 3 and a light chain variable region (VL) comprising an amino acid sequence having about 85%, about 90%, about 91%, about 92%, about 93%, about 94%, about 95%, about 96%, about 97%, about 98%, about 99%, or about 100% sequence identity to SEQ ID NO: 4.

In some aspects, the anti-Claudin-1 antibody comprises a VH comprising the amino acid sequence set forth in SEQ ID NO: 13; and a VL comprising the amino acid sequence set forth in SEQ ID NO: 14.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 13 and a light chain variable region (VL) comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 14.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 13 and a light chain variable region (VL) comprising an amino acid sequence having about 85%, about 90%, about 91%, about 92%, about 93%, about 94%, about 95%, about 96%, about 97%, about 98%, about 99%, or about 100% sequence identity to SEQ ID NO: 14.

In some aspects, the anti-Claudin-1 antibody comprises a complementary determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

In some aspects, the anti-Claudin-1 antibody comprises a complementary determining region (CDR) L1 comprising the amino acid sequence set forth in SEQ ID NO: 8, a CDR L2 comprising the amino acid sequence set forth as GAS, and a CDR L3 comprising the amino acid sequence set forth in SEQ ID NO: 10.

In some aspects, the anti-Claudin-1 antibody is administered intratumorally, intravenously, intraperitoneally, intramuscularly, intrathecally or subcutaneously.

In some aspects, provided herein is a method to reduce cholestasis, improve liver inflammation and function.

In some aspects, provided herein is a method to improve survival in cholangiopathies.

In some aspects, provided herein is a method to prevent cholangiocarcinoma.

### V. Administration

An anti-Claudin-1 antibody, or a biologically active fragment thereof, (optionally after formulation with one or more appropriate pharmaceutically acceptable carriers or excipients), in a desired dosage, can be administered to a subject in need thereof by any suitable route. Various delivery systems are known and can be used to administer antibodies, including tablets, capsules, injectable solutions, encapsulation in liposomes, microparticles, microcapsules, etc. Methods of administration include, but are not limited to, dermal, intradermal, intramuscular, intraperitoneal, intralesional, intravenous, subcutaneous, intranasal, pulmonary, epidural, and oral routes. An anti-Claudin-1 antibody, or a biologically active fragment thereof, or a pharmaceutical composition thereof, may be administered by any convenient or other appropriate route, for example, by infusion or bolus injection, by absorption through epithelial or mucosa linings (e.g., oral mucosa, bronchial mucosa, rectal and intestinal mucosa, etc.). Administration can be systemic or local.

In some aspects, the anti-Claudin-1 antibody is administered intratumorally, intravenously, intraperitoneally, intramuscularly, intrathecally or subcutaneously.

An anti-Claudin-1 antibody, or a biologically active fragment thereof, (optionally after formulation with one or more appropriate pharmaceutically acceptable carriers or excipients), will be administered in a dosage such that the amount delivered is effective for the intended purpose. The route of administration, formulation and dosage administered will depend on the therapeutic effect desired, the severity of the condition to be treated if already present, the presence of any infection, the age, sex, weight, and general health condition of the patient as well as upon the potency, bioavailability, and in vivo half-life of the antibody or composition used, the use (or not) of concomitant therapies, and other clinical factors. These factors are readily determinable by the attending physician in the course of the therapy. Alternatively or additionally, the dosage to be administered can be determined from studies using animal models (e.g., non-human primates or rodents). Adjusting the dose to achieve maximal efficacy based on these or other methods are well known in the art and are within the capabilities of trained physicians. As studies are conducted using anti-Claudin-1 antibodies, further information will emerge regarding the appropriate dosage levels and duration of treatment.

A treatment according to the present invention may consist of a single dose or multiple doses. Thus, administration of an anti-Claudin-1 antibody, or a biologically active fragment thereof, (or a pharmaceutical composition thereof) may be constant for a certain period of time or periodic and at specific intervals, e.g., hourly, daily, weekly (or at some other multiple day interval), monthly, yearly (e.g., in a time release form). Alternatively, the delivery may occur at multiple times during a given time period, e.g., two or more times per week; two or more times per month, and the like. The delivery may be continuous delivery for a period of time, e.g., intravenous delivery.

In general, the amount of anti-Claudin-1 antibody, or a biologically active fragment thereof, (or a pharmaceutical composition thereof) administered will preferably be in the range of about 1 ng/kg to about 100 mg/kg body weight of the subject, for example, between about 100 ng/kg and about 50 mg/kg body weight of the subject; or between about 1 μg/kg and about 10 mg/kg body weight of the subject, or between about 100 μg/kg and about 1 mg/kg body weight of the subject.

### VI. Pharmaceutical Compositions

As mentioned above, anti-Claudin-1 antibodies (and related molecules) can be administered per se or as a pharmaceutical composition. Accordingly, the present invention provides pharmaceutical compositions comprising an effective amount of an anti-Claudin-1 antibody, or a biologically active fragment thereof, described herein and at least one pharmaceutically acceptable carrier or excipient.

In some aspects, provided herein is a pharmaceutical composition for the treatment of a cholangiopathy, comprising a therapeutically effective amount of an anti-Claudin-1 antibody.

In some aspects, the cholangiopathy is Primary Sclerosing Cholangitis (PSC).

In some aspects, the cholangiopathy is Primary Biliary Cirrhosis (PBC).

In some aspects, the cholangiopathy is a genetic cholangiopathy. In some aspects, the cholangiopathy is an idiopathic cholangiopathy. In some aspects, the cholangiopathy is a malignant cholangiopathy. In some aspects, the cholangiopathy is a secondary sclerosing cholangitis.

In some aspects, the genetic cholangiopathies are selected from the group consisting of Alagille syndrome, Caroli syndrome, cystic fibrosis, and polycystic liver disease.

In some aspects, the idiopathic cholangiopathies are selected from the group consisting of autoimmune cholangitis, biliary atresia, idiopathic childhood or adulthood ductopenia, IgG4-associated cholangitis, primary biliary cirrhosis, and primary sclerosing cholangitis.

In some aspects, the malignant cholangiopathy is cholangiocarcinoma.

In some aspects, the secondary sclerosing cholangitis is selected from the group consisting of ABCB4 deficiency, abdominal trauma (surgical or blunt), AIDS cholangiopathy, amyloidosis, chemical/drugs (i.e., 5-fluorouracil), choledocholithiasis, eosinophilic or mast cell cholangitis, graft-vs-host disease involving the liver, iatrogenic biliary strictures, portal hypertensive biliopathy, recurrent pyogenic cholangitis, sarcoidosis, sickle cell disease, and vascular/ischemic (i.e., hepatic artery stenosis after liver transplant).

The pharmaceutical compositions may be administered in any amount and using any route of administration effective for achieving the desired prophylactic and/or therapeutic effect. The optimal pharmaceutical formulation can be varied depending upon the route of administration and desired dosage. Such formulations may influence the physical state, stability, rate of in vivo release, and rate of in vivo clearance of the administered active ingredient.

The pharmaceutical compositions of the present invention may be formulated in dosage unit form for ease of administration and uniformity of dosage. It will be understood, however, that the total daily dosage of the compositions will be decided by the attending physician within the scope of sound medical judgement.

### VII. Kits

In another aspect, the present invention provides a pharmaceutical pack or kit comprising one or more containers (e.g., vials, ampoules, test tubes, flasks or bottles) containing one or more ingredients of an inventive pharmaceutical composition, allowing administration of an anti-Claudin-1 antibody, or a biologically active fragment thereof.

Different ingredients of a pharmaceutical pack or kit may be supplied in a solid (e.g., lyophilized) or liquid form. Each ingredient will generally be suitable as aliquoted in its respective container or provided in a concentrated form. Pharmaceutical packs or kits may include media for the reconstitution of lyophilized ingredients. Individual containers of the kits will preferably be maintained in close confinement for commercial sale.

Optionally associated with the container(s) can be a notice or package insert in the form prescribed by a governmental agency regulating the manufacture, use or sale of pharmaceutical or biological products, which notice reflects approval by the agency of manufacture, use or sale for human administration. The notice of package insert may contain instructions for use of a pharmaceutical composition according to methods of treatment disclosed herein.

An identifier, e.g., a bar code, radio frequency, ID tags, etc., may be present in or on the kit. The identifier can be used, for example, to uniquely identify the kit for purposes of quality control, inventory control, tracking movement between workstations, etc.

In some aspects, provided herein is a kit for treating a subject suffering from a cholangiopathy, comprising a therapeutically effective amount of an anti-Claudin-1 antibody and an insert comprising instructions for use of the kit.

In some aspects, the cholangiopathy is Primary Sclerosing Cholangitis (PSC).

In some aspects, the cholangiopathy is Primary Biliary Cirrhosis (PBC).

In some aspects, the cholangiopathy is a genetic cholangiopathy. In some aspects, the cholangiopathy is an idiopathic cholangiopathy. In some aspects, the cholangiopathy is a malignant cholangiopathy. In some aspects, the cholangiopathy is a secondary sclerosing cholangitis.

In some aspects, the genetic cholangiopathies are selected from the group consisting of Alagille syndrome, Caroli syndrome, cystic fibrosis, and polycystic liver disease.

In some aspects, the idiopathic cholangiopathies are selected from the group consisting of autoimmune cholangitis, biliary atresia, idiopathic childhood or adulthood ductopenia, IgG4-associated cholangitis, primary biliary cirrhosis, and primary sclerosing cholangitis.

In some aspects, the malignant cholangiopathy is cholangiocarcinoma.

In some aspects, the secondary sclerosing cholangitis is selected from the group consisting of ABCB4 deficiency, abdominal trauma (surgical or blunt), AIDS cholangiopathy, amyloidosis, chemical/drugs (i.e., 5-fluorouracil), choledocholithiasis, eosinophilic or mast cell cholangitis, graft-vs-host disease involving the liver, iatrogenic biliary strictures, portal hypertensive biliopathy, recurrent pyogenic cholangitis, sarcoidosis, sickle cell disease, and vascular/ischemic (i.e., hepatic artery stenosis after liver transplant).

### Examples

The following examples are illustrative and do not limit the scope of the claimed aspects.

### Example 1. Improvement of Cholestasis and Fibrosis by Treatment with a CLDN1-Specific Monoclonal Antibody in the DDC Mouse Model for Cholangiopathies

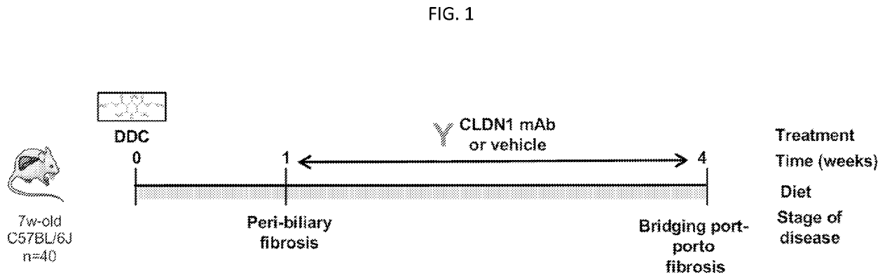

Proof-of-concept studies using CLDN1-specific mAbs were performed in the 3,5-Diethoxycarbonyl-1,4-Dihydrocollidine (DDC) mouse model, a well-recognized animal model for human cholangiopathies including PSC and PBC in which DDC was supplemented in the diet of C57BL/6 mice at 0.1% (FIG. 1). DDC impairs heme metabolism in murine hepatocytes, leading to insoluble crystals of protoporphyrin IX being secreted into the bile canaliculi, which causes bile flow obstructions. Within 3-6 weeks, the DDC diet causes cholestasis, inflammation, fibrosis, and activation of hepatic progenitor cells. Fibrosis was assessed by collagen and fibronectin staining of liver tissues.

All experiments were performed at the animal facility of Inserm U1110 according to local laws, ethics committee approval, and authorization by the French Ministry of Research and Higher Education. Forty 7-week-old C57BL/6J mice (Charles River Laboratories, France) were fed a 0.1%-DDC-supplemented diet (SAFE, Augy, France) for 4 weeks. After the first week, when peri-biliary fibrosis was established, mice were randomized into groups receiving weekly i.p. injection of CLDN1-specific H3L3 mAb (25 mg/kg, n=20) or vehicle control (n=20) for 3 weeks (a total of three Ab injections). Mice were sacrificed at the end of week 4 of diet and plasma and livers were harvested for subsequent analyses (FIG. 1). Fibrosis content was assessed by Sirius Red staining of formalin-fixed paraffin-embedded slices of whole liver lobes. Quantification of positive area was performed using the threshold method in ImageJ v1.53c software (National Institutes of Health, Bethesda, USA).

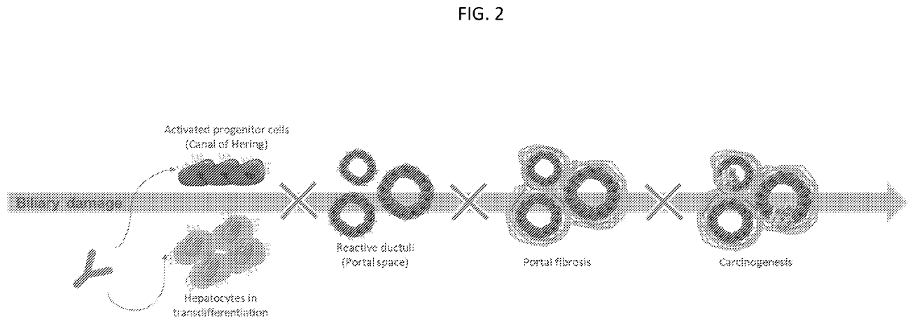

DDC impairs heme metabolism in murine hepatocytes and the insoluble crystals of protoporphyrin IX are secreted into bile canaliculi resulting in bile flow obstruction. CLDN1 expression in biliary cells, reactive ductules and surrounding hepatocytes identified the cells as therapeutic targets of the CLDN1 mAb (FIG. 2). Based on CLDN1 expression and functional data in the DDC model, treatment with anti-CLDN1 antibody likely ameliorates the phenotype of injured hepatocytes, bile duct cells or reactive ductular cells, leading to reduced fibrosis, improvement of cholestasis and prevention of carcinogenesis. Furthermore, anti-CLDN1 antibody treatment likely leads to restoration of a physiological healing response rather than “maladaptive regeneration”.

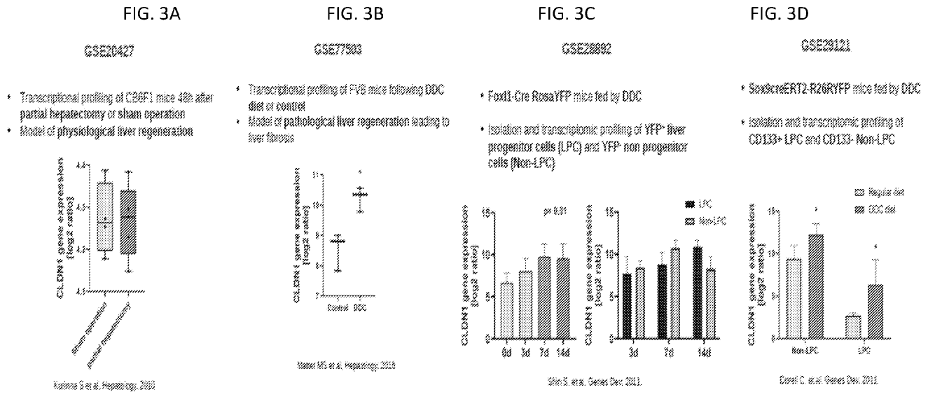

First, CLDN1 expression was analyzed in the DDC mouse model using publicly available RNA-Seq databases. As shown in FIGS. 3A-3D, CLDN1 was overexpressed in both progenitor cells (PROM1+) and non-progenitor cells in mouse models of regeneration, while it is not overexpressed in a model of physiological regeneration involving expansion and activation of progenitors, nor fibrosis deposition.

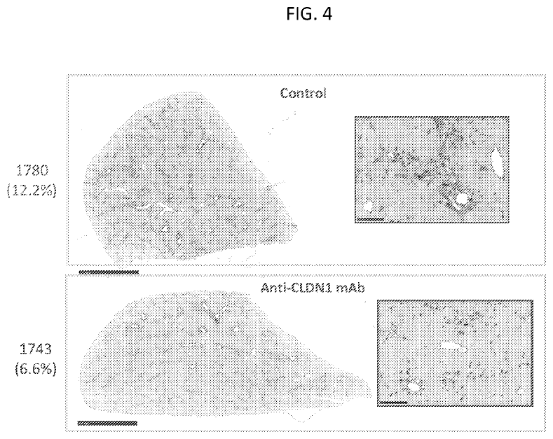

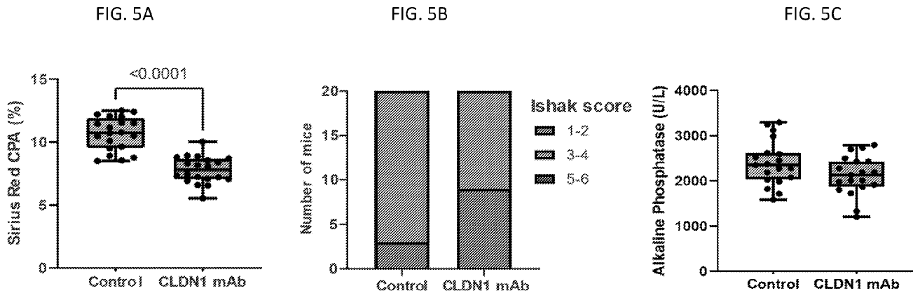

In an in vivo proof-of-concept study, the diseased animal was treated with CLDN1-specific mAb. 7-week-old C57BL/6J mice were fed a 0.1% DDC-supplemented diet for 4 weeks. After 1 week, when peri-biliary fibrosis was already established, mice were randomized into groups receiving weekly i.p injection of CLDN1 mAb (25 mg/kg, n=20) or vehicle control (n=20) for 3 weeks. Mice were sacrificed after 4 weeks of 0.1% DDC diet and three weeks of mAb treatment. Livers were harvested for subsequent analyses. CLDN1 mAb treatment resulted in decrease of cholestasis, a hallmark of PSC as indicated by a decrease of plasma alkaline phosphatase (2398 U/L vs 2119 U/L; FIG. 5C). Sirius red staining (FIG. 4) and automated analysis of the collagen proportionate area (CPA) (FIG. 5A) revealed significantly reduced fibrosis in CLDN1 mAb treated DDC mice by 26.8% versus vehicle control treated mice (p<0.0001, t-test). The total CPA was 7.79% in CLDN1 mAb treated mice vs 10.66% in vehicle controls; t-test p<0.0001; FIG. 5A) underscoring the robustness of the efficacy. Moreover, determination of the Ishak score as one of the most relevant fibrosis scoring systems applied in patients, confirmed significantly improved fibrosis in mice treated with CLDN1 mAb (FIG. 5B). Finally, major adverse effects or increased mortality in CLDN1 mAb treated versus vehicle control animals was not observed.

These results suggested a functional role of CLDN1 in the pathogenesis of PSC and provide robust preclinical proof-of-concept for a CLDN1-specific mAb as a novel therapeutic approach for PSC.

To investigate whether the effect of CLDN1 mAb on biliary fibrosis was dose-dependent, the experiment was repeated by performing a single ascending dose study using anti-CLDN1 mAb ALE-F02 (Roehlen et al. 2022) in the DDC model (Fickert et al (2007)). Since CLDN1 mAb ALE-F02 selectively binds to human CLDN1, a humanized transgenic mouse model was used in which mice were engineered to express a humanized extracellular loop 1 (h/mCLDN1 Tg mice) allowing robust binding by ALE-F02 CLDN1 mAb to CLDN1 in mouse tissues. Compared to the previous study shown in FIG. 1, the DDC feeding scheme was modified to comply with ethical requirements on weight loss.

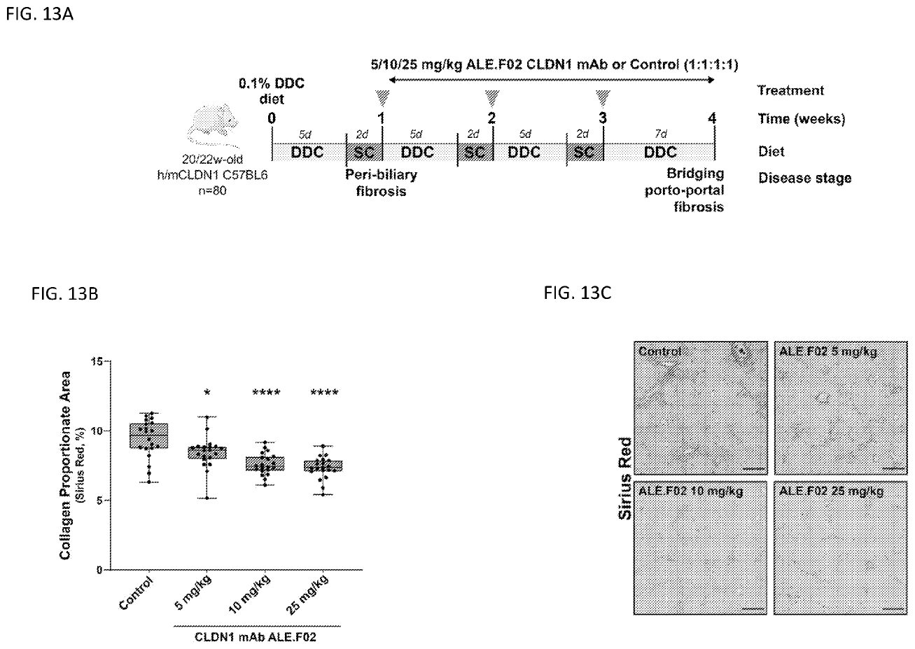

Eighty 20-22-week-old h/mCLDN1 Tg mice were fed a 0.1% DDC-supplemented diet for 5 days a week during the first three weeks, while fed a standard chow for 2 days a week. During the fourth week, mice were fed a DDC-supplemented diet for 7 days. Once peribiliary fibrosis has been established in week 1 (FIG. 13), mice were randomly assigned to four groups (1:1:1:1) to receive weekly either 5, 10 or 25 mg/kg ALE.F02 CLDN1 mAb or vehicle control (PBS) by intraperitoneal injection (FIG. 13). The total study duration was four weeks. At sacrifice, livers were harvested for subsequent analyses.

Automated analysis of the collagen proportionate area (CPA) (FIG. 13B) of Sirius Red-stained livers (FIG. 13C) revealed significantly reduced fibrosis in CLDN1 ALE.F02 mAb treated versus vehicle control-treated mice in a dose-dependent manner. Relative reductions of CPA were −10.3% for ALE.F02 5 mg/kg vs control (p=0.016, Mann-Whitney U test), −19.2% for ALE.F02 10 mg/kg vs control (p<0.0001, Student's t-test) and −21.9% for ALE.F02 25 mg/kg vs control (p<0.0001, Student's t-test).

These results show that treatment with ALE.F02 anti-CLDN1 mAb dose-dependently reduces biliary fibrosis in a state-of-the-art mouse model for cholangiopathies and biliary fibrosis. Given the presence of similar mechanism of biliary fibrosis in the DDC model and patients with cholangiopathies such as PBC and PSC, it is likely that treatment with CLDN1 specific Abs will improve biliary fibrosis in patients with cholangiopathies.

### Example 2. CLDN1 Expression in Patient Liver Tissues

Human liver tissues, fixed in formaldehyde and embedded in paraffin, were selected from the files of the Service of Pathology, University Hospital Geneva. These include cirrhotic specimens were obtained from patients with hepatectomy performed during liver transplantation. For all specimens, standard analysis using light microscopy was performed. The research was performed according to the Helsinki's declaration principles. Immunohistochemistry was performed as follows: after antigen heat retrieval, 3 μm sections of the formaldehyde-fixed, paraffin-embedded specimens were incubated with rabbit polyclonal anti-human Claudin-1 antibody (Elabscience, Texas, USA) at a 1:250 dilution 1 h at room temperature followed by an anti-rabbit antibody for 30 min (room temperature) and then liquid diaminobenzidine substrate-chromogen system (DakoCytomation, Glostrup, Denmark). For colocalization experiments, double immunostainings were performed on serial sections (3 μm thick) using Claudin-1 and each of these 5 different antibodies: rabbit polyclonal anti-human CK19 (ABCAM, Cambridge, UK) at a 1:400 dilution, rabbit polyclonal anti-human EPCAM (Invitrogen, Massachusetts, USA) at a 1:250 dilution. Briefly, 3 μm serial sections of paraffin-embedded kidneys were submitted to the appropriate antigen retrieval and incubated with Claudin-1 at a 1:250 dilution 1 hour at room temperature followed by an anti-rabbit antibody for 30 minutes (room temperature) and then liquid diaminobenzidine substrate-chromogen system (DakoCytomation, Glostrup, Denmark). Sections were then incubated with the adequate antibody for 1 hour at room temperature followed by the appropriate second antibody for 30 minutes and then by phosphatase alkaline-fast red enzyme system (DakoCytomation, Glostrup, Denmark). Counterstaining was performed using Mayer hematoxylin.

Data on CLDN1 gene expression in murine models were retrieved from bulk RNA-seq datasets deposited in Gene Expression Omnibus (GSE20427, GSE77503, GSE28892, GSE29121). This bulk RNA-seq data from murine models of liver regeneration revealed that CLDN1 is overexpressed by both PROM1+ liver progenitor cells and non-progenitor cells in the DDC mouse model (FIGS. 3A-3D). On the other hand, CLDN1 is not overexpressed in a model of physiological liver regeneration, i.e. partial hepatectomy (FIG. 3A).

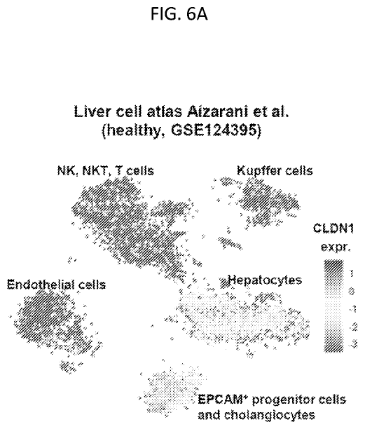

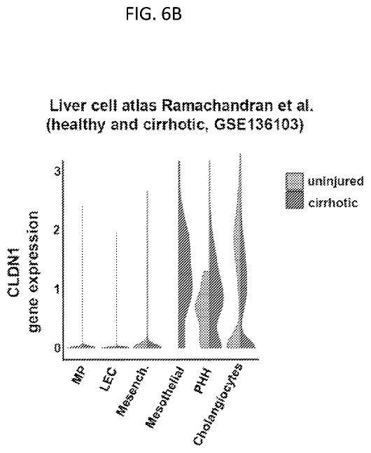

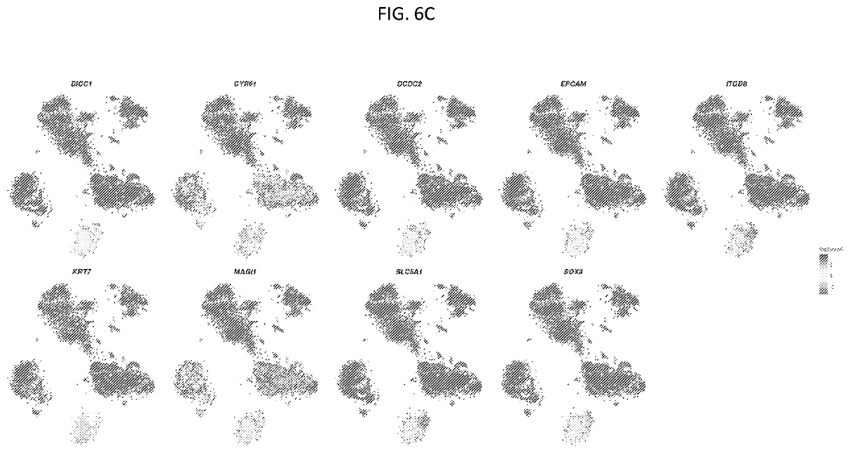

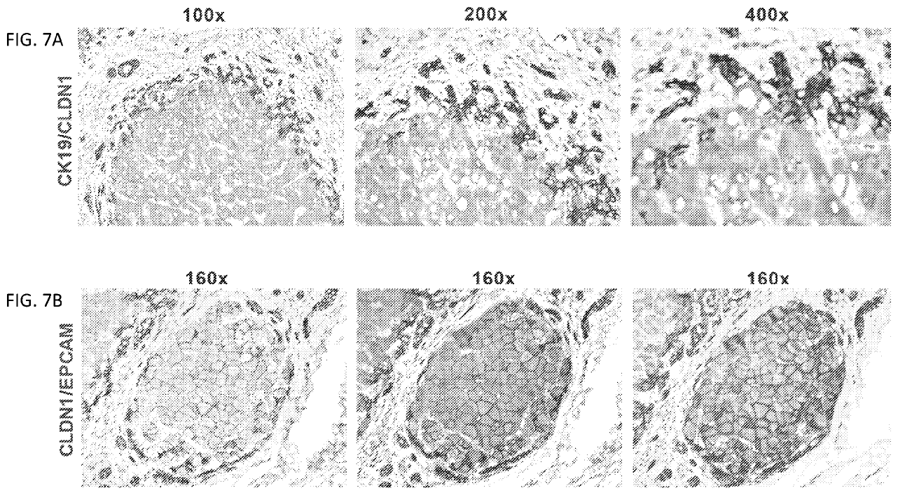

Data on CLDN1 expression in humans were retrieved from publicly available datasets of single cell-resolved transcriptomics studies, either from healthy (GSE124395) or fibrotic livers (GSE136103) based on original datasets deposited in GEO (Gene Expression Omnibus, NIH, Bethesda, USA. Single cell-resolved transcriptomic analyses of the human liver cell atlas and in the cirrhotic liver, revealed high level expression of CLDN1 in hepatocytes, EPCAM+ cells and cholangiocytes, as well as stellate cells and myofibroblasts (FIG. 6A). Moreover, CLDN1 expression co-clustered with known markers of biliary epithelial cells, i.e. bi-potent epithelial progenitor cells of the liver (FIG. 6B). The cells most highly expressing CLDN1 are also expressing markers of Biliary Epithelial Cells (Bipotent Liver Progenitor Cells), suggesting the association between high CLDN1 expression and a highly plastic cellular phenotype. (FIG. 6C). Confirming these observations on the protein level, double chromogenic staining of CLDN1/CK19 (FIG. 7A) and CLDN1/EPCAM (FIG. 7B) by immunohistochemistry revealed CLDN1 expression by ductular reactive cells, cholangiocytes and hepatocytes within a cirrhotic nodule. Using immunohistochemistry of patient tissues with advanced liver fibrosis we observed (1) co-expression of CLDN1 and CK19 in the surrounding ductular reaction as well as (2) co-expression of CLDN1 and EPCAM at the membrane of ductular reactive cells and hepatocytes (FIG. 7A-7B). Since these cell types have been shown to play a key role in the pathogenesis of cholangiopathies including PSC and PBC, CLDN1 is a therapeutic target for cholangiopathies.

To study the expression of CLDN1 as a target for therapeutic intervention across different cholangiopathies, CLDN1 expression was investigated in patients with cholangiopathies from transcriptomic data sets retrieved from public repositories. These include Gene Expression Omnibus (National Center for Biotechnology Information, NIH, Bethesda, MD; GSE46960, GSE206364, GSE118373) and Array Express (European Bioinformatics Institute, EMBL, Heidelberg, Germany; E-GEOD-61260).

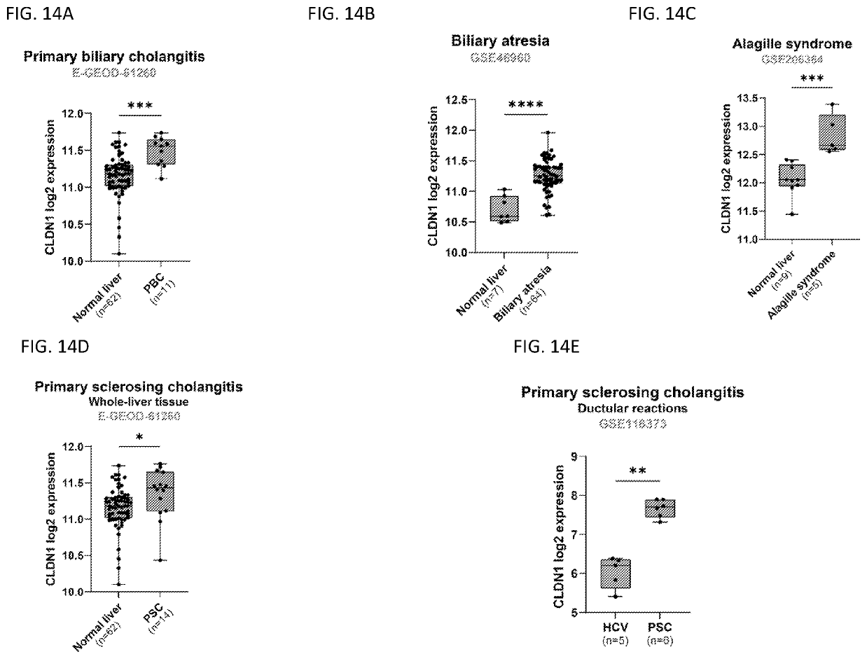

CLDN1 was robustly and significantly upregulated in the liver of patients with primary biliary cholangitis (p=0.0001, Mann-Whitney U test; E-GEOD-61260; FIG. 14A)(5), biliary atresia (p<0.0001, Mann-Whitney U test; GSE46960; FIG. 14B) (Bessho, K., et al., Hepatology 60(1):211-223 (2014)), the Alagille syndrome (p=0.001, Mann-Whitney U test; GSE206364; FIG. 14C) (Kriegermeier, A., et al., PLoS One 17(12):e0279016 (2022)) and primary sclerosing cholangitis (p=0.018, Mann-Whitney U test, E-GEOD-61260; FIG. 14D)(Bessho et al. 2014). Moreover, CLDN1 was among the top 20 upregulated genes in laser-microdissected ductular reactions of patients with primary sclerosing cholangitis versus ductular reactions of HCV-associated liver disease (p=0.004, Mann-Whitney U test; GSE118373; FIG. 14E) (Govaere, O., et al., J Pathol. 248(1):66-76 (2019)).

These data show that CLDN1 is strongly upregulated across multiple cholangiopathies, confirming its role as therapeutic target for CLDN1-specific mAbs across a large number of diverse and different cholangiopathies including for example PBC, PSC, biliary atresia and the Alagille syndrome.

### Example 3. CLDN1 in DDC Model in Human Liver Chimeric Mice

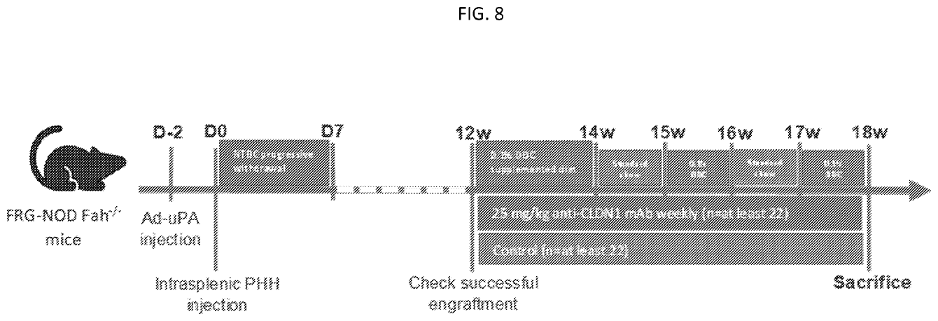

FRG-NOD Fah−/− mice will be used as an additional proof of concept for non-junctional CLDN-1 specific monoclonal antibody in a humanized model (FIG. 8). Primary Human Hepatocytes will be injected on Day 0. At 12 weeks post injection, mice will be checked for successful engraftment and humanization. If humanization is successful, mice will receive a pulsed DDC diet and weekly injections of anti-CLDN-1 monoclonal antibody (25 mg/kg) or control. Mice will be sacrificed after 12-20 weeks. After sacrifice, mice will be studied for fibrosis, ductular reaction, cholestasis, biliary metaplasia, and plasticity of human progenitor cells via standard techniques in the art such as immunohistochemistry.

### Example 4. CLDN1 as a Target in Proof-of-Concept Studies in the Biliary Ligation Model: Treatment with Anti-CLDN1 mAb H3L3 Ameliorates Survival, Cholestasis, Liver Function and Fibrosis

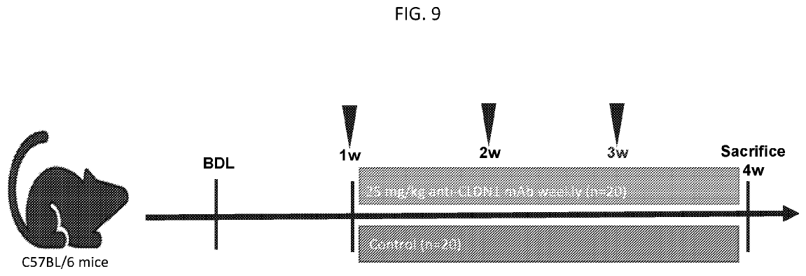

To investigate whether CLDN1 is a therapeutic target for cholangiopathies in general, the bile duct ligation (BDL) model was applied (FIG. 9). In the BDL model mice undergo surgical ligation of the common bile duct. Biliary obstruction leads to cholangiopathy and subsequent liver disease including fibrosis and inflammation. Complete interruption of bile flow causes acute cholestasis, with subsequent activation of liver progenitor cells and biliary fibrosis (Georgiev, P., et al., Br J Surg 95(5):646-656 (2008); Takahashi, Y., et al., Chapter 13—Animal Models of Liver Diseases. In: Conn P M, editor. Animal Models for the Study of Human Disease (Second Edition) Academic Press; p. 313-339 (2017)), Despite a high mortality, the rapid induction of biliary disease results in frequent use of this model as a universal model for cholangiopathies including PBC, PSC, biliary atresia and other cholangiopathies (Fickert et al., 2014).

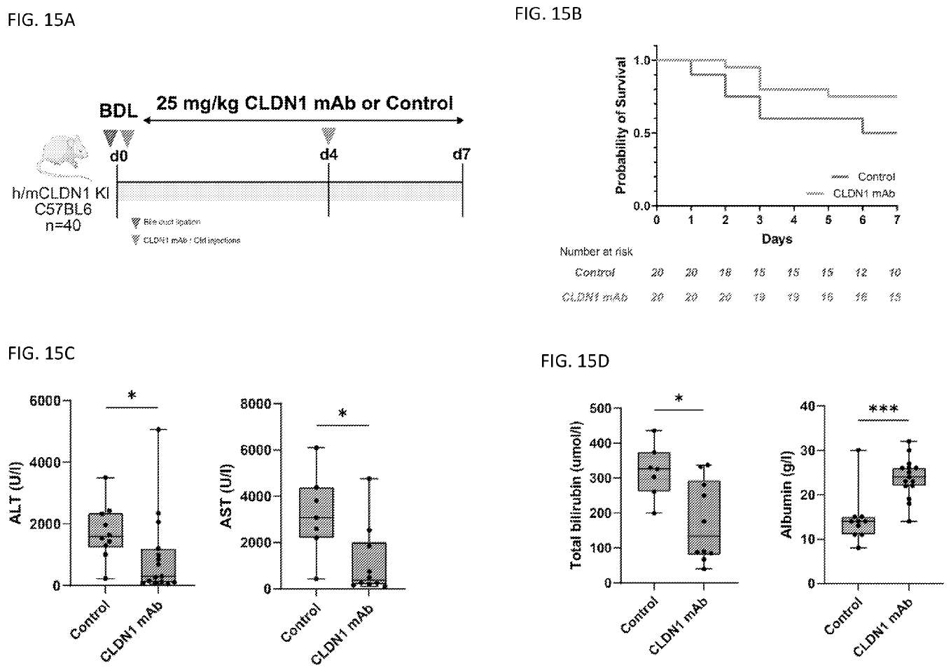

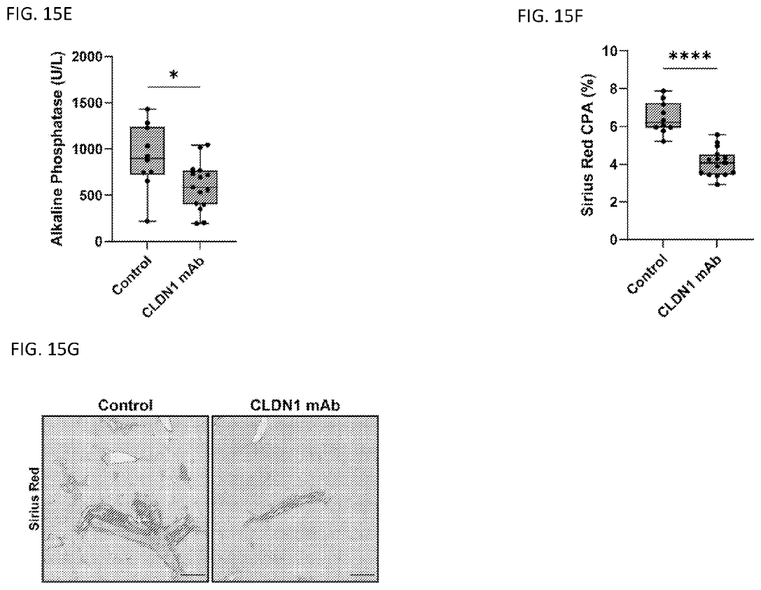

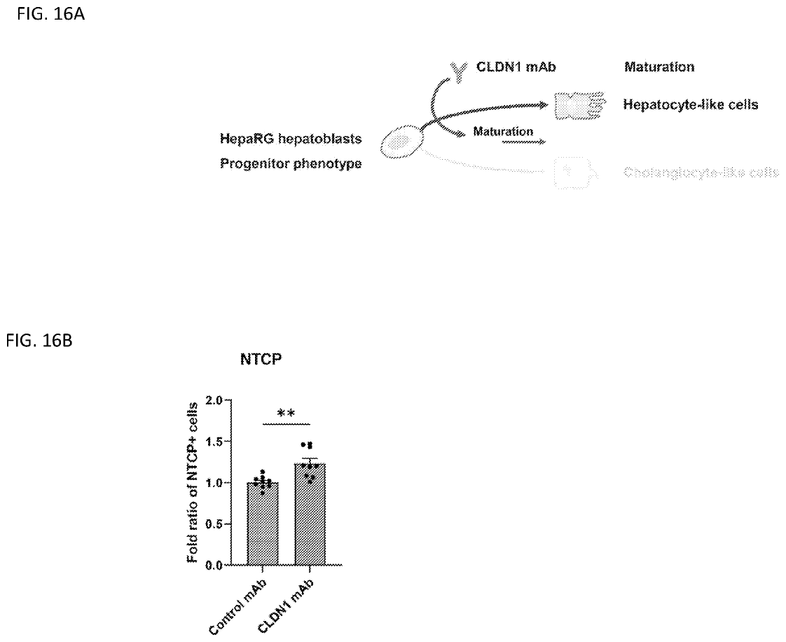

Transgenic mice expressing the human extracellular loop 1 of CLDN1 were used to study the efficacy of anti-CLDN1 mAbs in the BDL model. Taking into account the mortality rate of mice after surgical ligation of the common bile duct in this model, the protocol was shortened to a duration of 7 days after surgical ligation of the common bile duct. Forty mice underwent bile duct ligation, and were then assigned 1:1 to two treatment groups receiving either anti-CLDN1 mAb H3L3 (Colpitts et al. 2018) or vehicle control (PBS) intraperitoneally. The respective treatment was administered immediately after surgery and on day 4 after surgery (FIG. 15A). Surviving mice (n=10 in the control group; n=15 in the H3L3 group) were euthanized on day 7 after surgery, and livers and plasma harvested for subsequent analyses. Analysis of Kaplan-Meier survival curves showed a survival advantage in the treatment group (HR for death in the treatment group=0.39, 95% CI 0.14-1.15; p=0.08) (FIG. 15B). Liver function tests revealed a marked and significant improvement of markers of liver injury, liver function, and cholestasis in CLDN1 H3L3 mAb versus control-treated animals. These include markedly and significantly reduced levels of alanine aminotransferase ALT (306 vs 1591 U/l; −80.8% relative change; p=0.016, Mann-Whitney U test) and aspartate aminotransferase AST (372 vs 3069 U/l; −87.9% relative change; p=0.019, Mann-Whitney U test) (FIG. 15C) as markers for hepatocyte injury and inflammation; markedly and significantly reduced levels of total bilirubin (174.6 vs 318.4 μmol/l; −45.2% relative change; p=0.012, Student's t-test) (FIG. 15D) and markedly and alkaline phosphatase (598.4 vs 913.9 U/l; −34.5% relative change; p=0.017, Student's t-test) demonstrating marked and significant improvement of cholestasis. Furthermore, albumin levels increased in CLDN1 mAb versus control treated animals showing a markedly and significantly improved liver function by CLDN1 mAb treatment (24 vs 14 g/l; +71.4% relative change; p=0.0006, Mann-Whitney U test) (FIG. 15E). Automated analysis of the collagen proportionate area (CPA) (FIG. 15F) of Sirius Red-stained livers revealed marked and significant reduction of liver fibrosis in the liver CLDN1 mAb versus control treated mice (4.08% vs 6.45%; −36.8% relative change; p<0.0001, Student's t-test).

Collectively, these results demonstrate that anti-CLDN1 mAb treatment ameliorates survival and markedly and significantly improves liver function, cholestasis, and biliary fibrosis in a state-of-the-art animal model for obstructive cholestasis and cholangiopathies. Given the presence of similar mechanism of disease biology in the bile duct ligation model and patients with cholangiopathies, it is likely that treatment with CLDN1 specific Abs will improve biliary and liver function as well as overall outcome in patients with cholangiopathies.

### Example 5. CLDN1 mAb Treatment in a Long-Term Model of PSC and Progression to Cholangiocarcinoma

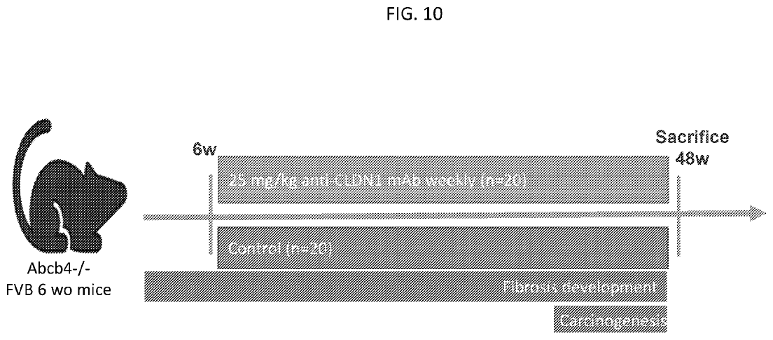

MDR2 −/− (Abcb4 −/−) mice will be used to model PSC and Cholangiocarcinoma (CCA) (FIG. 10). Mdr2(Abcb4) is a mouse ortholog of human MDR3 (ABCB4) gene encoding for the canalicular phospholipid transporter. Genetic disruption of the Mdr2 gene in mice causes a complete absence of phosphatidylcholine from bile, leading to liver fibrosis, sclerosing cholangitis and cholelithiasis. Mdr2−/− (Abcb4−/− mice) spontaneously develop biliary fibrosis recapitulating key PSC histological features and carcinogenesis over the course of 10-12 months. 6 weeks into the study, mice will begin receiving weekly injections of anti-CLDN-1 monoclonal antibody (25 mg/kg) or control. At 48 weeks, mice will be sacrificed. After sacrifice, mice will be studied for fibrosis, ductular reaction, and plasticity of human progenitor cells via standard techniques in the art such as immunohistochemistry.

### Example 6. CLDN1 mAb Effect on HepaRG Cells as Model for Cell Fate: Treatment with Anti-CLDN1 mAb H3L3 Promotes Maturation of Progenitor Cells into Hepatocyte-Like or Cholangiocyte-Like Cells

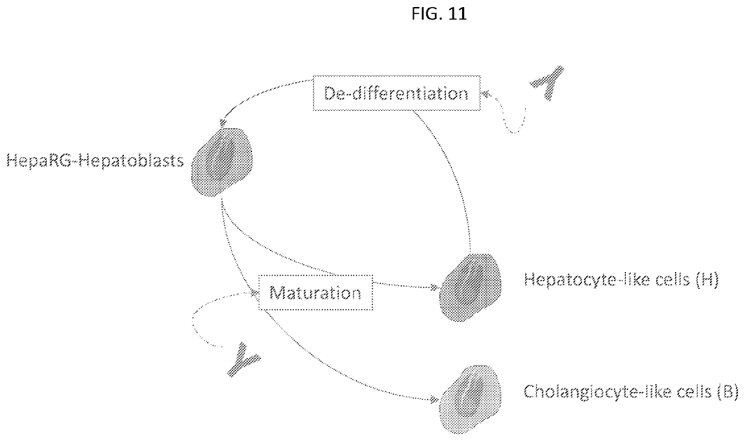

The HepaRG cell line, which is a hepatoblast cell line with dual differentiation potential towards hepatocytes or cholangiocyte-like cells, will be treated with anti-CLDN1 monoclonal antibodies (FIG. 11).

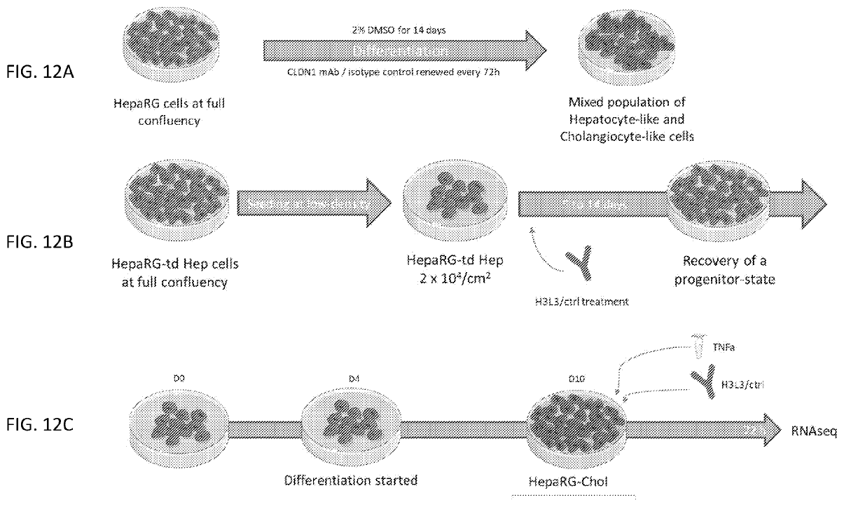

In a first experiment, HepaRG cells will be grown to full confluence, treated with 2% DMSO for 14 days, along with either anti-CLDN1 monoclonal antibodies or control every 3 days, resulting in a mixed population of hepatocyte-like and cholangiocyte-like cells. Fluorescence activated cell sorting (FACS) will be used to determine a ratio of hepatocyte-like v. cholangiocyte-like cells (FIG. 12A).

In a second experiment, HepaRG cells at full confluency will be reseeded at low-density (2×10⁴/cm²). After reseeding, cells were treated with anti-CLDN1 monoclonal antibody (H3L3) or control for 5 to 14 days. After treatment, cells will be harvested and analyzed for efficiency of de-differentiation back to the progenitor-state by assessing the expression of CK19 via FACS or immunofluorescence (FIG. 12B).

In a third experiment, HepaRG cells will be seeded on day 0 and differentiated into cholangiocyte-like cells by day 10. On day 10, CLDN1 expression will be confirmed via FACS. The cholangiocyte-like cells will then be treated with TNF alpha or anti-CLDN1 monoclonal antibodies for 72 hours. After 72 hours, the cells will be harvested and RNA-sequencing will be performed on the two populations (FIG. 12C).

It is expected for all three experiments that less pro-fibrotic and less pro-carcinogenic phenotypes will be observed following treatment with anti-CLDN1 monoclonal antibody.

### Example 7. Treatment with CLDN1 mAbs Inhibits Inflammatory, Fibrogenic and Carcinogenic Signaling Observed in PSC Patients in a Cholangiocyte Model

To unravel the cell circuits and signaling pathways targeted by CLDN1 mAb treatment in cholangiopathies, we analyzed transcriptional signatures of inflammatory-, fibrosis-, and carcinogenesis-related signaling in a cholangiocyte cell-based model and compared the antibody-induced modulations with changes in gene expression observed in the livers of patients with PSC. The cholangiocyte model consisted of HepaRG-differentiated cholangiocyte-like cells as described previously (Dianat, N., et al., Hepatology 60(2):700-714 (2014)) and incubated with tumor necrosis factor α (TNF-α). Gene expression of treated cells was assessed by RNASeq and compared with gene expression and signaling pathways in the liver of PSC versus control patients described in (Horvath et al. 2014)(E-GEOD-61260).

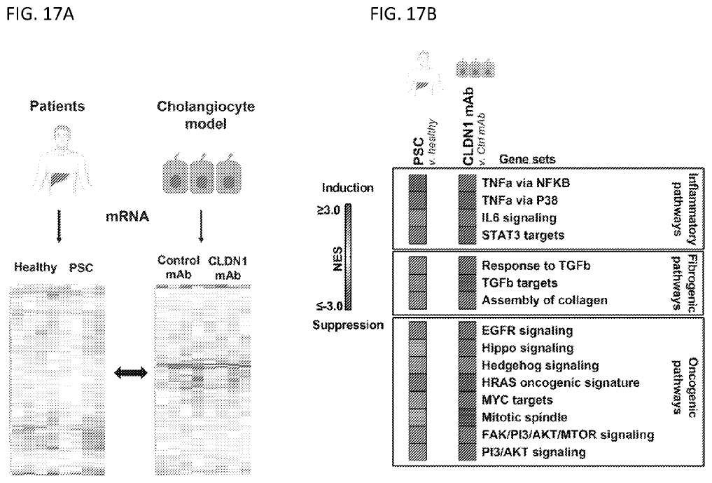

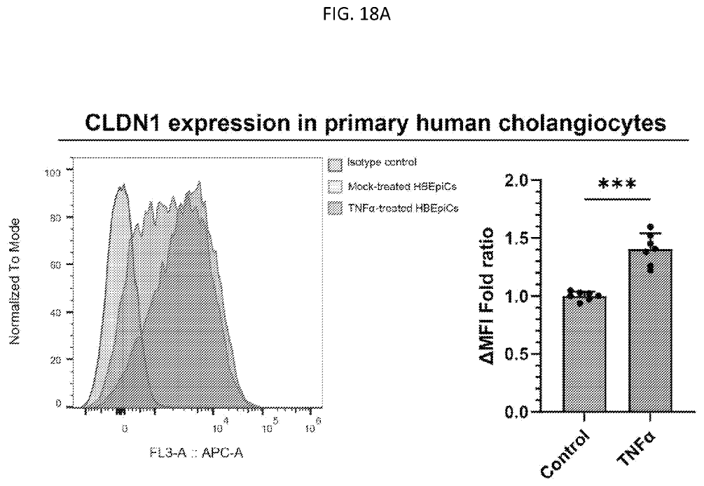

HepaRG progenitor cells were cultured in William's E medium supplemented with 2% FBS, 1 μg/ml insulin, and 1 μM hydrocortisone hemisuccinate as previously described (Dianat et al. 2014). To induce cholangiocyte differentiation, on day 4 after seeding cells were treated with IL-6 10 ng/ml for 2 days, then with 10 nM sodium taurocholate hydrate for 2 days, and then with 10 nM sodium taurocholate hydrate and 1.8 μM sodium butyrate for 2 days. After completion of the cholangiocytic differentiation, cells were treated with either 10 ng/ml TNF-α and 10 μg/ml H3L3 (Colpitts et al. 2018) anti-CLDN1 mAb or 10 ng/ml TNF-α and 10 μg/ml isotype control antibody for 96 hours. After 4 days, cells were harvested, and RNA extracted for RNAseq. Gene Set Expression Analysis (GSEA) was used for unbiased pathway analysis using Molecular Signature Database (MSigDB) (Subramanian, A., et al., Proc Natl Acad Sci USA 102(43):15545-15550 (2005)). Unbiased assessment of gene sets included in the MSigDB was also used for analyses of clinically observed gene expression changes in PSC liver tissue microarray data (E-GEOD-61260)(Horvath et al. 2014), which were then compared with RNA-Seq gene expression in the cholangiocyte-like cell-based model treated with CLDN1-specific or isotype control mAb. FDR <0.05 was considered as statistically significant (FIG. 17A).

As shown in FIG. 17B, in the cholangiocyte-like cell-based model, H3L3 CLDN1 mAb treatment reversed gene expression of a wide range of signaling pathways upregulated in the diseased livers of PSC patients. These included inflammatory pathways with the most pronounced inhibitory effects of CLDN1 versus control Ab observed on TNF-α-NFkB and STAT3 gene expression pathways (FIG. 17B). Moreover, gene expression of the pro-fibrogenic TGFβ signaling pathway, which was highly upregulated in PSC patient livers, was downregulated by CLDN1 mAb treatment in the cell-based model (FIG. 17B). Interestingly, several oncogenic signaling pathways, highly upregulated in the livers of PSC patients, were also downregulated in CLDN1 versus isotype mAb treated cells. These pathways included EGFR, HRAS, AKT/MTOR signaling (FIG. 17B).

Collectively, these data show that CLDN1 mAb treatment potently inhibited the expression of inflammatory, fibrogenic, and oncogenic signaling pathways in a biliary cell-based model, which were upregulated in liver tissues of PSC patients. Furthermore, these data show that the inhibitory effects of CLDN1 mAb treatment on inflammatory, pro-fibrogenic and pro-carcinogenic pathways are likely of clinical relevance in patients with PSC and cholangiopathies. These data suggest that treatment with CLDN1 mAbs will result in improvement of biliary disease, inflammation, fibrosis and carcinogenesis in patients with cholangiopathies.

### Example 8. TNFα Induces Upregulation of CLDN1 Protein Expression Targeted by CLDN1 mAbs and Treatment with CLDN1 mAb Inhibits TNFα-NFkB Signaling

To understand the regulation of CLDN1 expression as a target for CLDN1 mAbs in patient cholangiocytes and biliary disease, CLDN1 expression was studied in a cell-based model consisting of primary human cholangiocytes and treated with tumor necrosis factor alpha (TNFα) to model biliary inflammation and injury. TNFα was chosen as an inducer of inflammatory responses, since it was identified as a clinically relevant pro-inflammatory cytokine in sera of PSC patients (Zweers, S J., et al. Liver In. 36(9):1370-1377 (2016)). Moreover, NFkB (nuclear factor kappa-light-chain-enhancer of activated B cells) signaling—the downstream effector pathway induced by TNFα, was identified among the top upregulated signaling pathways in PSC livers as shown in FIG. 17B.

Primary human cholangiocytes were purchased from Innoprot, Bizkaia, Spain (Human Biliary Epithelial Cells, HBEpiC, ref. P10654; Batch #21614) and cultured on poly-1-lysine-coated surfaces according to manufacturer's instructions. Following stimulation, with 10 ng/ml TNFα for 24 hours, cells were harvested for assessment of CLDN1 expression by flow cytometry using anti-CLDN1 mAb H3L3 (Colpitts et al. 2018) and an anti-human AF647-conjugated secondary antibody.

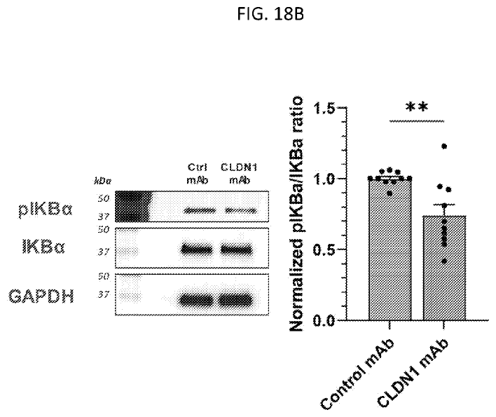

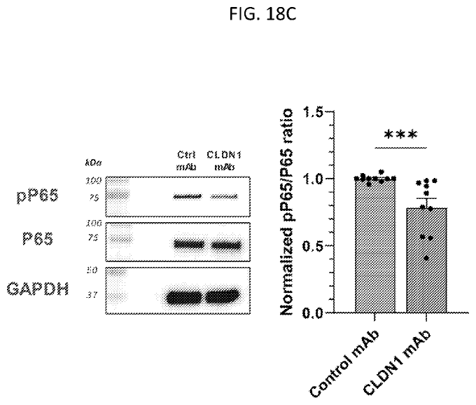

As shown in FIG. 18A, TNFα-stimulation significantly increased CLDN1 protein expression on primary human cholangiocytes (p=0.0006, Mann-Whitney U test) which strongly bound CLDN1 mAb H3L3 on the cholangiocyte cell surface as demonstrated by flow cytometry. These data show that CLDN1 expression is upregulated by defined pro-inflammatory cytokines and confirm the targetability for CLDN1 by CLDN1-specific mAbs on human biliary cells in inflammatory biliary disease such as PSC and cholangiopathies.

To investigate the effect of CLDN1 mAb H3L3 on pro-inflammatory signaling in biliary cells, NFκB signaling was studied in HBEpiC primary human cholangiocytes. Cholangiocytes were treated with either CLDN1 H3L3 (Colpitts et al. (2018)) or an isotype control mAb (both at 10 μg/ml) for 72 hours, with antibodies renewed after 48 hours. NFκB signaling was induced by treatment with TNFα (10 ng/ml) in the presence of the antibody treatment. 60 or 120 minutes post incubation with TNFα, cells were harvested for protein extraction and immunoblotting for IKBα (nuclear factor of kappa light polypeptide gene enhancer in B-cells inhibitor), pIKBα (phosphorylated nuclear factor of kappa light polypeptide gene enhancer in B-cells inhibitor), P65 and pP65 (phosphorylated P65). GAPDH staining was used as loading control. Protein signals were then quantified by imaging. As shown in FIG. 18B-C, CLDN1 mAb H3L3 treatment inhibited TNF-α-mediated phosphorylation of IKBα (p=0.003, Mann-Whitney U test, n=10, FIG. 18B) and P65 (p=0.0002, Mann-Whitney U test, n=10, FIG. 18C) in primary human cholangiocytes. These data show that treatment with CLDN1 versus control mAbs inhibits TNFα-NFκB signaling in a cell-based model of human cholangiocytes. The CLDN1 antibody-inhibition of TNFα-NFκB signaling may contribute to its therapeutic effect in cholangiopathies in patients.

### Example 9—CLDN1 mAb Effect on HepaRG Cells as Model for Cell Fate: Treatment with Anti-CLDN1 mAb H3L3 Promotes Maturation of Progenitor Cells into Hepatocyte-Like Cells

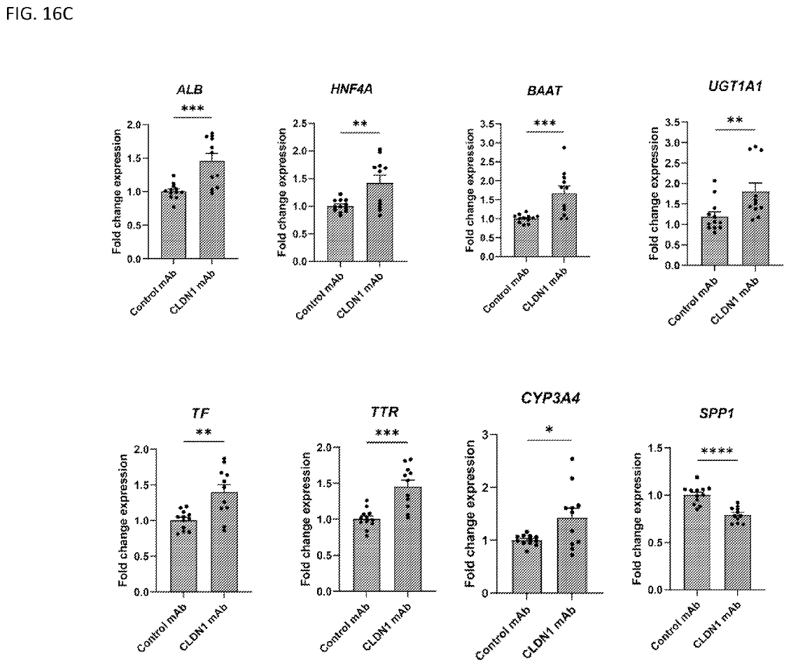

To further understand the mechanism of action of CLDN1 treatment in the improvement of liver function in biliary diseases, the effect of CLDN1 mAbs was investigated using a liver progenitor cell model based on HepaRG cells (Laurent, V., et al., Methods Mol Biol. 987:295-314 (2013)). The HepaRG progenitor model consists of hepatoblast-like cells with dual differentiation potential towards hepatocytes (Marion, M J, et al., Methods Mol Biol. 640:261-272 (2010)) or cholangiocytes (Marion et al. 2010). Using a defined protocol, HepaRG can be differentiated to hepatocyte-like cells (Marion et al. 2010) allowing to study the effect of compounds on the hepatocyte maturation process. Here, the HepaRG model was used to study the effect of CLDN1 H3L3 versus isotype control mAb treatment on differentiation into of HepaRG hepatoblasts progenitor cells into hepatocyte-like cells (FIG. 16A).

HepaRG progenitor cells were cultured as previously described (Laurent et al. 2013). After growing to full confluence for 14 days, cells were treated with 2% DMSO to induce hepatocyte differentiation. anti-CLDN1 mAb (10 μg/ml) (Colpitts et al. 2018) or an isotype control (10 μg/ml) were added to the differentiation medium. New antibody was added during medium replacement twice a week. After two weeks of differentiation, cells were harvested for protein and gene expression markers of mature hepatocytes. The abundance of mature hepatocyte-like cells was assessed by flow cytometry using a fluorescent-conjugated pre-S1 HBV peptide, specifically binding Na+-taurocholate cotransporting polypeptide (NTCP) protein on target cells (Ni, Y., et al. Methods Mol Biol. 1540:15-25 (2017))—a marker of mature hepatocytes. Gene expression of typical hepatocyte and ductal markers was assessed by RT-qPCR.

CLDN1 mAb treatment of HepaRG cells markedly increased the abundance of NTCP+ cells (n=3 independent experiments, p=0.002, Student's t-test) (FIG. 16B), indicating that anti-CLDN1 mAb treatment promotes maturation of progenitor cells into hepatocytes (Ni Y., et al. 2017). Furthermore, as shown in FIG. 16C, anti-CLDN1 mAb treatment upregulated expression of hepatocyte markers albumin (ALB)(n=4 independent experiments, p=0.0004, Student's t-test), hepatocytic nuclear factor 4 alpha (HNF4A) (n=4 independent experiments, p=0.005, Student's t-test), cytochrome P450 3A4 (CYP3A4) (n=4 independent experiments, p=0.018, Student's t-test), transthyretin (TTR) (n=4 independent experiments, p=0.0001, Student's t-test), UDP glucuronosyltransferase family 1 member A1 (UGT1A1) (n=4 independent experiments, p=0.007, Mann-Whitney U test); bile acid-CoA:amino acid N-acyltransferase (BAAT)(p=0.0009, Student's t-test) and transferrin (TF) (n=4 independent experiments, p=0.0016, Student's t-test). In contrast, expression secreted phosphoprotein 1 (SPP1), a marker of immature cells or ductal marker was significantly downregulated (p<0.0001, Student's t-test).

These results show that anti-CLDN1 mAb treatment significantly upregulates expression of genes associated with a broad range of hepatocyte-specific functions, including hepatocyte-specific protein biosynthesis, bile acid transport and metabolism as well as xenobiotic metabolism. The down-regulation of SPP1 gene expression further confirms that CLDN1 mAb treatment modulates cell plasticity with a shift from an immature towards a mature hepatocytic fate. Collectively, these findings show that anti-CLDN1 mAb treatment promotes maturation of immature hepatic progenitor cells into mature hepatocyte-like cells in a cell-based model which may explain in part its effect on improvement of liver function as shown in the DDC and bile-duct ligation animal models, respectively. Given that these mechanisms have been shown to play a functional role in patients, it is likely that these observations are clinically relevant and may contribute to improvement of liver disease in patients with cholangiopathies treated with CLDN1-specific Abs.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

Any examples provided herein are offered by way of illustration and not by way of limitation.

1. A method of treating a cholangiopathy in a human subject in need thereof, comprising administering a therapeutically effective amount of an anti-Claudin-1 antibody to the human subject, wherein the anti-Claudin-1 antibody comprises a heavy chain variable domain complementary determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7, and a light chain variable domain complementary determining region (CDR) L1 comprising the amino acid sequence set forth in SEQ ID NO: 8, a CDR L2 comprising the amino acid sequence GAS, and a CDR L3 comprising the amino acid sequence set forth in SEQ ID NO: 10.

2. (canceled)

3. (canceled)

4. A method of reducing Biliary Fibrosis, comprising administering an anti-Claudin-1 antibody to the human subject, wherein the anti-Claudin-1 antibody comprises a heavy chain variable domain complementary determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7, and a light chain variable domain complementary determining region (CDR) L1 comprising the amino acid sequence set forth in SEQ ID NO: 8, a CDR L2 comprising the amino acid sequence GAS, and a CDR L3 comprising the amino acid sequence set forth in SEQ ID NO: 10.

5. The method of claim 1, wherein the cholangiopathy is Primary Sclerosing Cholangitis (PSC).

6. The method of claim 1, wherein the cholangiopathy is Primary Biliary Cirrhosis (PBC).

7.-9. (canceled)

10. The method of claim 1, wherein the anti-Claudin-1 antibody is humanized.

11. The method of claim 1, wherein the anti-Claudin-1 antibody comprises a VH comprising the amino acid sequence set forth in SEQ ID NO: 3 or SEQ ID NO: 13.

12. The method of claim 1, wherein the anti-Claudin-1 antibody comprises a VL comprising the amino acid sequence set forth in SEQ ID NO: 4 or SEQ ID NO: 14.

13. The method of claim 1, wherein the anti-Claudin-1 antibody comprises a VH comprising the amino acid sequence set forth in SEQ ID NO: 3; and a VL comprising the amino acid sequence set forth in SEQ ID NO: 4.

14. The method of claim 1, wherein the anti-Claudin-1 antibody comprises a VH comprising the amino acid sequence set forth in SEQ ID NO: 13; and a VL comprising the amino acid sequence set forth in SEQ ID NO: 14.

15. The method of claim 1, wherein the anti-Claudin-1 antibody is administered intratumorally, intravenously, intraperitoneally, intramuscularly, intrathecally or subcutaneously.

16.-28. (canceled)

29. The method of claim 1, wherein the cholangiopathy is a biliary fibrosis.

30. The method of claim 29, wherein the biliary fibrosis is caused by cystic fibrosis.

31. The method of claim 29, wherein the biliary fibrosis is caused by an IgG4-related disease.

32.-34. (canceled)

35. The method of claim 1, wherein the cholangiopathy is biliary atresia.

36. The method of claim 1, wherein the cholangiopathy is Alagille syndrome.

37. (canceled)

38. (canceled)

39. A method of reducing cholestasis in a human subject in need thereof, comprising administering a therapeutically effective amount of an anti-Claudin-1 antibody to the human subject, wherein the anti-Claudin-1 antibody comprises a heavy chain variable domain complementary determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7, and a light chain variable domain complementary determining region (CDR) L1 comprising the amino acid sequence set forth in SEQ ID NO: 8, a CDR L2 comprising the amino acid sequence GAS, and a CDR L3 comprising the amino acid sequence set forth in SEQ ID NO: 10.

40. (canceled)

41. (canceled)
